(19) United States
(12) Patent Application Publication
(10) Pub. No.: US 2010/0329994 A1
(43) Pub. Date: Dec. 30, 2010

(54) Use of Deuterium Dioxide for Treating Hyperproliferative Skin Diseases

(76) Inventor: Thomas Bayerl, London (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/446,237

(22) PCT Filed: Oct. 18, 2007
(86) PCT No.: PCT/DE2007/001855

§ 371 Date: May 30, 2009

(30) Foreign Application Priority Data
Oct. 18, 2006 (DE) 10 2006 049 185.8

(51) Int. Cl. A61K 33/00; A61K 9/12; A61P 17/00; A61P 17/02; A61P 17/06; A61P 17/10; A61P 17/12

(52) U.S. Cl. 424/43; 424/600

(57) ABSTRACT

The invention relates to D₂O and the use thereof for producing a medicament for the prophylaxis and/or treatment of hyperproliferative skin diseases. The invention also relates to plasters, bandages, aerosols and formulations.

The present invention relates to the use of deuterium dioxide (D₂O) for the prophylaxis and/or therapy of hyper-proliferative skin disorders. The invention further relates to D₂O-containing patches, bandages, aerosols and formulations for targeted topical application of D₂O to the skin.

Hyperproliferative disorders in mammalian tissue are distinguished in general by cell growth being above average by comparison with normally proliferating cells, with an increased cell division rate associated therewith. Virtually all types of tissues and cells can be affected by hyperproliferative disorders, such as hepatic, pulmonary, retinal, intestinal, pancreatic and cutaneous tissues and cells. In the case of hyperproliferative skin disorders, a distinction is made between malignant disorders, which include in particular all cancers such as, for example, melanoma, basal-cell carcinoma, squamous eptithelial carcinoma, small-cell carcinomas, adenocarcinomas, large-cell carcinomas, adenosquamous carcinomas, sarcomatoid carcinomas, mucoepidermoid carcinomas, adenoid-cystic carcinomas and epithelial-myoepithelial carcinomas and non-malignant disorders such as, for example, in particular psoriasis, e.g. psoriasis vulgaris, psoriasis pustulosa and psoriasis-arthritis, keratoses, e.g. benign lichenoid keratosis, palmoplantar keratosis, follicular keratosis, verruca seborrhoica and lichen planus-like keratosis, acne, e.g. acne vulgaris and acne inversa, porokeratoses, e.g. disseminated porokeratosis, porokeratosis of Mibelli, punctate porokeratosis, linear porokeratosis and disseminated porokeratosis, diabetic foot syndrome and further keratonizing skin diseases. In addition, scars of the skin are also in some cases, for example hypertrophic scars and keloids, the result of hyperproliferation of the connective tissue of the skin which is manifested in particular by increased formation of fibroblasts, dermal collagen and keratinocytes. Scar (lat. cicatrix) generally refers to a low-quality, fiber-rich replacement tissue representing the final state of wound healing.

A hypertrophic scar arises shortly after wound healing or even during its course and is an erythematous thickening of the skin which remains restricted to the area of the original wound, projects above the level of the skin, is often toric and spontaneously, but often not completely, regresses. The cause is hyperproliferation of connective tissue cells. The occurrence of hypertrophic scars is increased if a wound is not immobilized or protected or if additional infection occurs. The keloid is distinguished from the hypertrophic scar. A keloid is an excessive scarring also outside the original wound and represents a benign tumor which is based on excessive growth of fibroblasts, projects above the skin, may occur in particular after injuries, operations or else spontaneously, and is to be regarded as an impaired healing process. Keloids show only a small tendency to regress and arise on a genetic basis. Hypertrophic scars and keloids are included in the present invention among the non-malignant hypertrophic skin disorders.

Psoriasis represents a widespread non-malignant hyperproliferative skin disorder. In Germany alone, about 2% of the population, which corresponds to about 1.5 million people, are affected by psoriasis. The latter is a chronic, non-infectious disorder of the skin. Whereas the predisposition to this disorder is inherited, the triggers are, however, external, for example stress. Among the various forms of psoriasis, divided into mild, intermediate and severe psoriasis, about 90% of all cases are of psoriasis vulgaris, and rarer cases are psoriasis pustulosa and psoriasis-arthritis. The complex skin changes occurring during psoriasis are characterized by 3 factors:
a) typical epidermal changes,
b) typical changes in the cutaneous vascular system and
c) a characteristically inflammatory infiltrate.

In psoriasis there is in particular hyperproliferation of keratinocytes with altered differentiation, characterized by parahyperkeratosis, aberrant expression of keratin 6/16, involucrin, filaggrin, and integrin adhesion molecules (VLA-3, VLA-5, VLA-6, a6b4). In addition, the keratinocytes express de novo MHC class II and ICAM-1 molecules. These are molecules which mediate the interaction with leukocytes. Endothelial activation in psoriatic skin leads to vasodilatation, angiogenesis and increased expression of MHC class II, ICAM-1, E-selectin, VCAM-1 and other receptors. Finally, there is in psoriatic lesions an infiltrate of activated lymphocytes in dermis and epidermis, neutrophils in the dermis and in epidermal Munro microabscesses, and mast cells and macrophages. T lymphocytes in particular play a primary role in the pathogenesis of psoriatic skin changes. This view is supported by the association of psoriasis with certain MHC alleles, the therapeutic effect of T-cell-suppressive substances such as cyclosporine A or the lymphocyte-specific toxin DAB389IL-2, the linkage of a gene which is expressed to an increased extent in psoriasis patients with an interleukin IL-2-regulating gene, the healing of psoriatic lesions after bone marrow grafts and the response to a therapy with CD4-specific antibodies. In some cases, an oligoclonal expansion has been detected as an indication of antigen-specific reactions, but it has not as yet been possible to identify reliably an autoantigen.

Neutrophilic granulocytes also play an important role in the pathogenesis of psoriasis. It is assumed that neutrophils are stimulated by the interaction of chemotactic factors to migrate into psoriatic lesions. GM-CSF, which is expressed to an increased extent in psoriatic lesions, is able to induce the integrin aMb2 on neutrophils. The latter then bind to ligands (for example ICAM-1) on endothelial and activated epidermal cells. The observation that Munro microabscesses are located almost exclusively in parakeratotic areas led to the conjecture that neutrophils are able to influence keratinocyte differentiation. The stimulated (activated) neutrophilic granulocytes produce reactive oxygen free radicals and proteases which might influence keratinocyte proliferation, demask antigens, activate complement or degrade tissue constituents. Finally, neutrophils may stimulate DNA synthesis in keratinocytes through secretion of various lipid mediators. It was possible to confirm a pathogenic puncture of neutrophilic granulocytes in psoriatic skin changes in the mouse model. The psoriatic tissue changes are mediated by a network of different cytokines. These are supplemented by numerous chemokines which play an essential role in particular for the tissue-specific localization of leukocytes.

The role of tissue-specific leukocyte localization mediated by adhesion receptors in psoriasis is, by contrast, only now starting to become known and is based on the interplay of various molecular interactions. On extravasation, leukocytes initially enter into transient bindings with endothelial cells which are mediated by selectins. “Rolling” leukocytes are able to bind through b2-integrins firmly to ligands from the immunoglobulin superfamily. Firm leukocyte-endothelium binding also involves b1-integrins and their ligands. If leukocytes have left the “vascular bed”, in particular b1-integrins bring about the binding to ECM proteins.

However, these numerous pieces in the puzzle of the pathogenesis of psoriasis do not permit a clear overall picture of this complex skin disease. The prophylaxis and/or therapy thereof is correspondingly difficult and even now does not show any satisfactory results, and in most cases psoriasis remains untreatable in the long term. The therapy of psoriasis is made difficult in particular by two essential factors:
firstly it is a chronically recurrent disorder which may require treatment over a very long period,
secondly it is necessary to take account of individual factors such as internal concomitant disorders (diabetes, hypertension, liver damage), as well as the clinical forms of psoriasis and their acuteness, the skin type (pigmentation) and pretreatment.

Psoriasis therapy generally takes place as local therapy or/and systemic therapy, and in the form of a phototherapy which can preferably be combined with other types of therapy (Lehmann P, Ruzicka T: Neue Entwicklungen in der Psoriasistherapie, Dt ärztebl 1996; 93: A-3188-3193).

In local antipsoriatic therapy, anthralin (dithranol, cignolin) is still even now a much used medicament especially for patients treated in hospital and in day clinics. It is initially applied in low concentrations to the skin, and the concentration is then increased stepwise. The therapy can be carried out as long-term therapy or short-contact treatment. There are no restrictions on the duration of therapy or the affected surface of the body for anthralin. Local irritation (cignolin dermatitis) may occur at the outset or if the anthralin concentration is increased too quickly. A disadvantage of anthralin therapy, which occurs in particular with higher concentrations, is discoloration of surrounding skin (called anthralin brown, an oxidation product of the active ingredient). Topical therapy of psoriasis with analogs of vitamin D3 has gained a place especially in outpatient treatment in recent years (van de Kerkhof PC: An update on vitamin D3 analogues in the treatment of psoriasis. Skin Pharmacol Appl Skin Physiol 1998; 11: 2-10). The vitamin D3 analogs calcipotriol and tacalcitol which can be employed in various pharmaceutical forms are employed in particular for chronic stationary psoriasis (plaque type). The good antiproliferative and differentiation-inducing effect results in a reduction in the scaling and infiltration. Vitamin D products are therefore very suitable for combination with a UV-B phototherapy. The topical retinoid tazarotene is new in psoriasis therapy and therefore experience with this active ingredient is still limited. Topical corticosteroids are the most frequently used medicaments for the therapy of psoriasis. Corticosteroids have proved useful in particular for the inflammatory forms. Topical corticosteroids have, however, only short-term effects, so that recurrence of psoriatic foci is to be expected after discontinuation.

Systemic therapy is necessary whenever psoriasis affects large parts of the integument and the disease activity is high (frequent recurrences). In this connection, ciclosporin revealed in numerous studies very good activity for severe psoriasis vulgaris (Mrowietz U, Färber L, Henneicke von Zepelin H H, Bachmann H, Wetzel D, Christophers E: Long-term maintenance therapy with ciclosporine and posttreatment survey in severe psoriasis: results of a multicenter study. German Multicenter Study. J Am Acad Dermatol 1995; 33: 470-475). Pustular types of psoriasis (psoriasis pustulosa) and psoriasis-arthritis can also be treated successively with ciclosporin. Ciclosporin inhibits the activity of T cells, antigen-presenting cells and mast cells and thus influences essential effector cells of the psoriatic tissue reaction. However, a prominent unwanted side effect is a dose-dependent restriction of kidney function and the development of hypertension.

A further suitable systemic therapy of psoriasis is treatment with aromatic retinoids. The active ingredient approved in this connection inter alia is acitretin which has replaced the previously commercially available etretinate. Although acitretin monotherapy is not as effective for plaque-type psoriasis as other systemic medicaments, good treatment results can be achieved in pustular types of psoriasis (psoriasis pustulosa). An improved effect is possible through the (established) combination with PUVA (“Re-PUVA”). Unwanted side effects which may occur are skin dryness, diffuse alopecia, and bone and muscle pain. Possible biochemical findings are an increase in serum lipids and/or an increase in liver enzymes.

In addition, fumaric esters have been used for years for the therapy of severe psoriasis. The products currently approved for therapy, Fumaderm initial and Fumaderm, contain a plurality of esters of fumaric acid in varying concentration (Christophers E, Mrowietz U: Psoriasis, In: Freedberg I M, Eisen A Z, Wolff K, Austen K F, Goldsmith L A, Katz S I, Fitzpatrick T G, eds.: Dermatology in General Medicine, New York: McGraw-Hill, 1999; 495-521). Severe unwanted side effects are, however, in particular gastrointestinal symptoms and the occurrence of flush symptoms. On prolonged therapy, in addition a reduction in leukocyte counts with lymphopenia and eosinophilia may be observed.

Among the phototherapies, both UV-B and UV-A light have local immunosuppressant actions and lead to numerous effects, especially in the epidermis and dermis. In this connection, UV-A and UV-B light differ in particular through the “minimal erythema dose” (MED) necessary to achieve a response. Combination of UV A with a photosensitizer (PUVA therapy) shows as photochemotherapy the strongest antipsoriatic effect. PUVA therapy can be carried out as oral or as bath PUVA therapy. In conventional (oral) PUVA treatment, the photosensitizer (usually 8-methoxypsoralen) is taken orally. Absorption of the active ingredient and accumulation in the skin within two hours is followed by UV-A irradiation. Bath PUVA therapy has been particularly used in recent years and entails the photosensitizer being supplied to the skin via the bath water. The advantages are that the photosensitizer has no systemic effect and an overall lower total UV-A dose is necessary. It is also possible to use the photosensitizer in a suitable cream base for targeted therapy of localized psoriatic foci (“cream PUVA”). Moreover, a combination of salt baths (over five percent) with subsequent UV irradiation (brine phototherapy) has been widely used very recently.

It is additionally possible in particular for combinations of the aforementioned types of therapy to be very effective in the treatment of psoriasis (combination therapies). Combination of external (i.e. substances applied externally, such as ointments, creams and liquid active ingredients) with UV therapy or systemic medicaments, e.g. ciclosporin, acitretin, fumaric esters and methotrexate (MTX), usually leads for example to a substantially improved response of the psoriatic efflorescences and may also lead to a prolongation of the symptom-free time. Well known conventional combinations for this purpose are the use of strong brine baths with subsequent UV-B irradiation (brine phototherapy), topical therapy with corticosteroids or vitamin D3 analogs in conjunction with UV-B light or PUVA and combination of systemic retinoids with PUVA (Re-PUVA). However, it must also be taken into account that many systemic medicaments such as ciclosporin, acitretin, MTX (methotrexate) and fumaric esters cannot, because of severe side effects, be combined with simultaneous UV therapy.

Keratoses represent a significant group of non-malignant hyperproliferative skin disorders and are usually associated with an increased rate of division of skin cells, especially keratinocytes—hyperkeratosis. Hyperkeratosis does not, however, cause the disorder in all the disorders encompassed by the term “keratosis”, but does promote the development of these disorders and influences the development and the severity of the disorders. In this sense, the term “keratosis” encompasses besides actinic keratosis, epidermolytic hyperkeratosis, hyperkeratosis lenticularis perstans, keratosis pilaris ichthyoses, benign lichenoid keratosis, palmoplantar keratosis, follicular keratosis, verruca seborrhoica, lichen planus-like keratosis and porokeratosis, especially disseminated porokeratosis, porokeratosis of mibelli, punctate porokeratosis, linear porokeratosis, disseminated porokeratosis, also disorders such as acne, especially acne vulgaris, acne inversa (depending on the severity also called acne comedonica, acne papula-pustulosa and acne conglobata), hidradentis suppurativea, acne aestivalis, acne cosmetica, acne medicamentosa, acne venenata and acne tarda, which are associated with hyperkeratosis of the terminal follicles or a follicular hyperkeratosis of apocrine sweat glands (see, for example, Marks R. and Plewig G. (1988) Proceedings of an International Symposium on Acne and related disorders, Cardiff). Keratoses are moreover associated with a number of complications which occur in diabetes mellitus, so that the term keratosis also encompasses inter alia the diabetic foot syndrome.

Scars of the skin represent a further non-malignant hyperproliferative skin disorder of great importance. The therapies known in the prior art for scars of the skin, especially hyperproliferative scars and keloids, can be divided into the following therapeutic policies:
a) injection of corticoids,
b) surgical treatment (excision),
c) compression therapy,
d) radiation therapy,
e) laser therapy,
f) cryotherapy,
g) application of hypoallergenic microporous plasters (“tape strips”) by means of suitable adhesives and
h) application of silicone gel films.

A detailed review of the aforementioned types of therapy and their efficacy demonstrated by studies is given for example in the article by Ziegler et al. (Ziegler et al., “International clinical recommendations on scar management.” Plastic and Reconstructive Surgery, 2002, vol. 110, No. 2, 560-571). According to this, it was possible to demonstrate for each of these therapies and also for various combinations thereof an efficacy, but in most cases only for a limited period here too, since initially a (partial) recurrence of the treated scars of the skin was observed. In addition, a complete disappearance of the scars of the skin was reported only in a few cases. The use of silicone films is ascribed a particularly great potential for the preventive avoidance or prophylaxis and/or therapy of scars of the skin on the basis of randomized control studies. Combinations of the above-mentioned therapies with an active ingredient able to inhibit or limit hyperproliferation also appear to be a particularly effective therapeutic possibility. However, the active ingredient should if possible have only local effects here too, and its side effects on the system of the treated organism and on wound healing should be negligible.

However, none of the therapies described in the prior art for scars of the skin has a mechanism of action which acts on one of the essential bases of scar formation: the hyperproliferation of dermis and/or of keratinocytes or fibroblasts in the region of the scar.

In addition, a general problem of all the therapies mentioned in the prior art for hyperproliferative skin disorders is the lack of a long-term effect thereof and the severe side effects thereof. Ordinarily, all the known therapies lead merely to a time-limited, short symptom-free period of the disease. The cyclic repetitions of the therapy or combination therapies necessitated thereby lead to a permanent stress on the system with severe side effects in all cases. In addition, virtually all systemically administered medicaments, for example corticoids or cytostatics, cause severe side effects affecting the whole organism of the treated patient even with a short treatment time. These disadvantages of the prior art relate not only to the hyperproliferative skin disorders described in particular above, namely psoriasis and scars of the skin, specifically hypertrophic scars and keloids, but extend to all malignant and non-malignant hyperproliferative skin disorders. There is thus a pressing need for novel, improved active ingredients whose administration leads to only slight or negligible side effects, or none at all, and can thus be repeated as often as desired.

It is accordingly an object of the present invention to provide improved active ingredients for the treatment of hyperproliferative skin disorders. It is an additional object of the present invention to provide improved topical application techniques.

These objects are achieved by the present invention. The invention relates in its first two aspects to the use of deuterium dioxide (D₂O) for the prophylaxis and/or therapy of hyperproliferative skin disorders and to the use of D₂O for the manufacture of a medicament for the prophylaxis and/or therapy of hyperproliferative skin disorders, especially malignant disorders of the skin, such as melanoma, basal-cell carcinoma, squamous eptithelial carcinoma, small-cell carcinomas, adenocarcinomas, large-cell carcinomas, adenosquamous carcinomas, sarcomatoid carcinomas, mucoepidermoid carcinomas, adenoid-cystic carcinomas and epithelial-myoepithelial carcinomas, and non-malignant skin disorders such as psoriasis, e.g. psoriasis vulgaris, psoriasis guttata, psoriasis capitis, psoriasis inversa, psoriasis pustulosa and psoriasis-arthritis; keratoses such as, for example, actinic keratosis, epidermolytic hyperkeratosis, hyperkeratosis lenticularis perstans, keratosis pilaris ichthyoses, benign lichenoid keratosis, palmoplantar keratosis, follicular keratosis, verruca seborrhoica, lichen planus-like keratosis and porokeratoses, e.g. disseminated porokeratosis, porokeratosis of Mibelli, punctate porokeratosis, linear porokeratosis, disseminated (depending on the severity also called acne comedonica, acne papula-pustulosa and acne conglobata), hidradentis suppurativea, acne aestivalis, acne cosmetica, acne medicamentosa, acne venenata and acne tarda, hyperkeratoses in connection with diabetes mellitus, such as, for example, diabetic foot syndrome; and scars of the skin, especially hypertrophic scars and keloids.

The terms “prophylaxis” and/or “therapy” refer to every measure suitable for the treatment of a hyperproliferative skin disorder which relates either to a preventive treatment (prophylaxis) of such a disorder becoming manifest, or the symptoms thereof becoming manifest, or for preventing a recrudescence of such a disorder, for example after a completed therapeutic treatment period, or relates to treatment of the symptoms of such a disorder which has already appeared (therapy).

“Hyperproliferative cells” in the context of the present invention refer to cells capable of autonomous, abnormal growth, i.e. a rapidly proliferating growth. In this connection, pathological conditions, i.e. conditions representing a disorder, and non-pathological conditions, i.e. conditions representing a deviation from the normal cell proliferation rate, but not associated with a disorder of the tissue, e.g. increase in the cell division rate during wound healing, are to be understood. The hyperproliferative cells described herein of scars of the skin, especially hypertrophic scars and keloids, and the growth thereof are/is assigned to the pathological conditions.

“Hyperproliferative disorders” or “neoplastic disorders” of the skin refer in the context of the invention to malignant and non-malignant pathological conditions.

“Malignant” pathological conditions include inter alia all types of tumor- or cancer-like cell growth, oncogenic processes and malignant transformed cells, tissues or organs, e.g. carcinoma, sarcoma or metastases. Examples are melanoma, basal-cell carcinoma, squamous eptithelial carcinoma, small-cell carcinomas, adenocarcinomas, large-cell carcinomas, adenosquamous carcinomas, sarcomatoid carcinomas, mucoepidermoid carcinomas, adenoid-cystic carcinomas and epithelial-myoepithelial carcinomas.

The “non-malignant” disorders include in particular psoriasis, e.g. psoriasis vulgaris, psoriasis guttata, psoriasis inversa, psoriasis capitis, psoriasis pustulosa and psoriasis-arthritis; keratoses, e.g. benign lichenoid keratosis, palmoplantar keratosis, follicular keratosis, verruca seborrhoica and lichen planus-like keratosis, porokeratoses, e.g. disseminated porokeratosis, porokeratosis of Mibelli, punctate porokeratosis, linear porokeratosis and disseminated porokeratosis, acne, e.g. acne vulgaris, acne inversa (depending on the severity also called acne comedonica, acne papula-pustulosa and acne conglobata), hidradentis suppurativea, acne aestivalis, acne cosmetica, acne medicamentosa, acne venenata and acne tarda, and hyperkeratoses in connection with diabetes mellitus, such as, for example, the diabetic foot syndrome; and scars of the skin, especially hypertrophic scars and keloids.

As precondition for an effective prophylaxis and/or therapy of hyperproliferative skin disorders, an pharmaceutical active ingredient should—taking account of the mode of administration thereof—generally comply with the following requirements:
a) local usability through topical application over a period of any desired length (for pharmaceutical active ingredients to be applied topically);
b) substantially homogeneous distribution of the active ingredient in the region of the site of action, and the avoidance of excessive local concentrations;
c) preferential accumulation of the pharmaceutical active ingredient in the hyperproliferative areas of skin, connected with retardation of its transdermal transport into the system, i.e. into the blood vessels;
d) preferred action on hyperproliferative cells in the affected area of skin in the sense of a disproportion-ately great retardation, preferably inhibition, of the growth rate thereof compared with the surrounding non-hyperproliferative cells, and
e) substantial tolerance of the healthy cells in the skin tissue and in the whole system in relation to the pharmaceutical active ingredient in order to avoid side effects, especially also in terms of immune responses.

The use according to the invention of D₂O as pharmaceutical active ingredient—alone or in combination with at least one further pharmaceutical and/or at least one further non-pharmaceutical active ingredient—and the directed topical application thereof onto selected regions of the skin complies with all these requirements and additionally has a distinct advantage over the therapies and pharmaceutical active ingredients known in the above prior art in terms of its topical application, the directed local activity and, associated therewith, the avoidance of stress on the system (i.e. the bloodstream) of the treated organism. More details will be given of this below.

The present invention is accordingly based on the realization that deuterium dioxide, referred to as D₂O hereinafter, is an effective active ingredient with a potential long-term effect and suitable for long-term therapy of hyperproliferative skin disorders which reduces, preferably eliminates, the severe side effects known in the prior art. As already mentioned, hyperproliferative cells have a far higher cell division rate than normally proliferating cells. Since cells take up water, referred to as H₂O hereinafter, during division thereof, hyper-proliferative cells take up, owing to their increased cell proliferating cells. D₂O, frequently also referred to as “heavy water”, is a substance extremely similar to “natural water” H₂O and is taken up by dividing cells instead of (if only D₂O is available) or in parallel with H₂O (if D₂O and H₂O are available). The reasons for this are explained in detail below.

D₂O and H₂O differ physically through their replacement of the hydrogen atoms of H₂O by deuterium atoms, with D₂O having a density which is approximately 10% higher and a viscosity which is approximately 25% higher. In addition, the melting and boiling points of D₂O are higher than those of H₂O. A detailed comparison of the properties is given in the Handbook of Chemistry and Physics, section 6 (Handbook of Chemistry and Physics, David R. Lide, Editor, 79^{th} edition, 1998 CRC Press, Boca Raton, USA).

Whereas the physical differences between H₂O and D₂O are rather small, there are significant physiological differences (see inter alia Kushner D J et al., Pharmacological uses and perspectives of heavy water and denatured compounds, Can J Physiol Pharmacol. 1999 February; 77(2): 79-88). Thus, for example, it has been found that although many algae and bacteria are able to exist in 100% D₂O entirely normally for long periods, and this is possible with up to 70% D₂O for protozoa, this does not apply to animal cells. In this case, with D₂O concentrations in the organism of more than 20-25%, various enzymatically controlled reactions are increasingly altered, in particular inhibited. One reason for this is presumably the higher bond strength of the hydrogen bonds when the hydrogen atom of H₂O is replaced by a deuterium atom. This increased bond strength occurs both in aqueous solutions of H₂O and D₂O and in the bonding of water to organic molecules, and the effect appears to be even more pronounced in the case of organic molecules (Cuma M, Scheiner S, Influence of Isotopic Substitution on Strength of Hydrogen Bonds of Common Organic Groups, Journal of Physical Organic Chemistry, 1997 volume 10, 383-395).

An important aspect based on this altered bonding property is the realization that increased concentrations of D₂O may retard or entirely inhibit cell division. This probably takes place mainly through inhibition of DNA synthesis (Takeda H et al. Mechanisms of cytotoxic effects of heavy water (deuterium oxide: D₂O) on cancer cells, Anticancer Drugs. 1998 September; 9(8): 715-25) or of mitosis in the cycle of division of animal cells (Laissue J A et al. Survival of tumor-bearing mice exposed to heavy water or heavy water plus methotrexate, Cancer Research, 1982, Vol. 42, (3) 1125-1129).

As stated above,
D₂O has at a suitable concentration in a proliferating cell inter alia the ability to retard or inhibit cell division;
proliferating cells take up D₂O as well as H₂O; and hyperproliferative cells take up, owing to their increased cell division rate, disproportionately more D₂O than normally proliferating cells.
   As a result, D₂O accumulates through active or passive transport in the hyperproliferative cells in an animal tissue, preferably of a mammal, in which rarely or normally proliferating cells are present besides an area of hyperproliferating cells—instead of or in addition to H₂O, whereby the cell division rate thereof is disproportionately greatly slowed, retarded or even entirely inhibited. This effect is not only highly desired in the therapy of many medical indications of hyperproliferative malignant, such as, for example, tumor therapy, and non-malignant disorders, but is an aim which is greatly strived for, because hyper-proliferative cells bring about an uncontrolled and in most cases harmful growth in the tissue.

In the case of tumor growth in the large bowel and in the squamous epithelial cells in the mouth and pharynx, this effect of D₂O was demonstrated in the 1980s experimentally on Balb/c/-nu/nu mice (Alternatt H J et al., Heavy water delays growth of human carcinoma in nude mice; Cancer. 1988 Aug. 1; 62(3): 462-6). Experimental animals were supplied with drinking water which was enriched with 20-30% D₂O (based on the total volume of the drinking water). The detectable antitumor effect of D₂O in the drinking water extended only over the period of administration of this mixture of D₂O and H₂O water, however; no persistence of the antitumor effect was to be found after changing to normal H₂O administration.

An antitumor effect was also detectable in other tissues. Thus, recently published cell culture studies with 3 tumor cell lines from the pancreas likewise support an antitumor effect (Hartmann, J. et al., Effects of heavy water (D₂O) on human pancreatic tumor cells, Anticancer Res. 2005 September-October; 25(5): 3407-11). Other investigations yielded unambiguous results for the D₂O-mediated antitumor effect in tissues of neoplastic brain cells and suggest that D₂O can induce apoptosis in malignant astrocytoma cells (Uemura, T. et al., Experimental validation of deuterium oxide-mediated antitumoral activity as it relates to apoptosis in murine malignant astrocytoma cells, Neurosurg. 2002 May; 96(5): 900-8). The authors also concluded in this study that D₂O has a cytotoxic effect on tumor tissue and thus represents a cytostatic. Published studies in which an established cytostatic was administered together with D₂O instead of H₂O for the treatment of murine xenotransplanted tumors (Alternatt, H J, Heavy water (D₂O) inhibits growth of human xenotransplanted oropharyngeal cancers. An animal experiment study in nude mice, Laryngol Rhinol 0 to 1 (Stuttg). 1987 April; 66(4): 191-4) also confirm an additional antineoplastic effect of D₂O.

Nevertheless, a therapeutic benefit of the systemic administration of D₂O to an animal organism, especially of a mammal, for the treatment of hyperproliferative disorders has, despite the above-mentioned advantageous cytotoxic effect, been rejected to date in the prior art, because the D₂O concentrations necessary for such an effect are above 25% and thus severe side effects, for example an alteration in protein synthesis or protein folding, were expected (Kushner D J et al., Pharmacological uses and perspectives of heavy water and denatured compounds, Can J Physiol Pharmacol. 1999 February; 77(2): 79-88). In addition, all the studies which have been published in the prior art on the effect of D₂O on cells, organs and organisms conclude that the effect of D₂O persists only for the period of D₂O administration, and thus very long cycles of D₂O administration would be necessary. The result of this would in turn be a great burden of D₂O on the whole treated organism and the side effects associated therewith, as mentioned above. This is a further aspect of the reason why no therapies of hyperproliferative disorders with D₂O as active ingredient have been described in the prior art to date.

Accordingly, an effective prophylaxis and/or therapy of malignant or non-malignant hyperproliferative disorders overall, and especially of the skin, by systemic or non-systemic—for example topical application to the skin—administration of D₂O has not been described anywhere in the prior art to date.

However, it has now become possible according to the invention to apply the antineoplastic effect or antitumor effect of D₂O described in the prior art after systemic D₂O administration to an effective topical application of D₂O to the skin for the treatment of hyperproliferative skin disorders. The basis thereof is in particular the following unique properties of D₂O as pharmaceutical active ingredient which distinguishes it from all other pharmaceutical and in particular cytotoxic active ingredients:
1) the possibility of topical application of D₂O to the skin allows a D₂O concentration which is sufficiently high for therapeutic efficacy to be reached in the epidermis or dermis of the skin without other organs of the body needing to experience similarly high concentrations (which are possibly harmful for them) of D₂O. A crucial problem, discussed in the prior art, of achieving therapeutically effective D₂O concentrations at the site of action (usually over 20% D₂O based on the total water content of the cell) without severe side effects for other organs or healthy skin tissue is thus solved. The basis of this is the directed transport of D₂O through the stratum corneum of the skin to the epidermis or dermis;
2) the state of aggregation of D₂O on topical administration may be liquid, gaseous or solid, and transport into the skin can take place through direct contact of the active ingredient composition with the skin as well as indirectly by diffusion through an intermediate layer (e.g. air, porous membrane, polymer network);
3) especially in the case where pure liquid D₂O is administered alone (pure D₂O), it must be stated that D₂O has a unique advantage over all other liquid pharmaceutical active ingredients. It can be transported like normal water (H₂O) into the skin and, in addition, the depth of penetration of D₂O into the skin can be adapted, through the strength and direction of the osmotic gradient and a manipulation of these two variables, to the therapeutic objective;
4) the hydrogen bond strength of D₂O is, as already described above, higher than that of H₂O, especially in the bonding of water to organic molecules. Topically administered D₂O undergoes molecular bonding through hydrogen bonds to the nearest available cell surface and thereby displaces the H₂O deposited there because of its higher bond strength. The exchange frequency of the D₂O molecules with the H₂O surroundings is in turn, owing to this increased bond strength (and to the greater weight of the D₂O molecule), somewhat slower than for H₂O (König, S., et al., “Molecular dynamics of water in oriented multilayers studied by quasi-elastic neutron scattering and deuterium-NMR relaxation”, 1994, J. Chem. Phys. 100, 3307-3316). The result thereof is an increased probability of direct occupation of the cell surface by D₂O molecules. The result as a consequence is a preferred internalization of D₂O in the cell, and it is thus able on arrival in the cytoplasm to display its effect directed at inhibition of cell division. In the case of the skin, therefore, D₂O in fact accumulates in the region of the keratinocytes and is not released into the system with the rate of transport of H₂O. This is a step which is crucial for the activity, because only higher concentrations of D₂O in the cell in fact result in a reduction in cell division. Since hyper-proliferative cells (benign and malignant) usually have a higher permeability and uptake capacity for water than normal cells, it is ensured at the same time that the accumulation of D₂O in these cells is disproportionately greater than in normally proliferating cells, and cell growth is correspondingly reduced or inhibited there. Owing to these properties, topical application of D₂O to the skin is extremely valuable for the therapy of hyperproliferative disorders where the desired site of action is in the epidermis or at most in the dermis of the skin;
5) D₂O is the only non-radioactive molecule having very similar properties to H₂O. Cells in general, and especially keratinocytes of the skin, are unable to “distinguish” between the two molecules, and thus D₂O is transported into the cell, and reaches the cell nuclei, through active and passive transport in the same way as H₂O. Cell barriers of any types which prevent penetration of other pharmaceutical active ingredients are circumvented thereby, and likewise defense mechanisms at the cellular level, such as the internalization into lysosomes or the activation of MDR (multiple drug resistance) transporters or at the organ level by the immune system, each of which might reduce or inhibit the activity of the pharmaceutical active ingredient D₂O, are substantially eliminated;
6) a further advantage of D₂O as anti-hyperproliferative pharmaceutical active ingredient is the fact that concentrations below 20% D₂O (based on the total water content of the cell) display no significant effects in the cell, and thus normal cells which, owing to their water permeability and/or water uptake being lower than that of hyperproliferative cells, take up comparatively little D₂O are scarcely exposed to the effects of D₂O; and
7) hyperproliferative, non-malignant skin disorders such as psoriasis are distinguished by increased cell growth, especially of keratinocytes, with simultaneous infiltration of lymphocytes. Since D₂O is able to retard or inhibit the proliferation of cells, it is possible, because of the increased need for water of hyperproliferative cells such as keratinocytes, to achieve a differential effect on the proliferation thereof compared with the surrounding normally proliferating cells from non-psoriatic tissue. The same effect can be achieved in the case of the treatment of hypertrophic scars and keloids, because a hyperproliferation of in particular fibroblasts and dermal collagen cells in the case of scar formation is regarded as also being a cause of the scar tissue growing above the level of the skin.

The present invention likewise makes it possible through the topical application according to the invention to employ locally high, therapeutically effective D₂O concentrations on the skin and, at the same time, to reduce or even completely prevent the stresses on the system (i.e. of the blood stream) and the side effects on healthy skin tissue which is not to be treated, and on tissue of other organs (which are not to be treated) of the treated organism, such as, for example, of the liver or kidney, (which may be caused by a high concentration of D₂O of more than 20% D₂O based on the total water content of the cell). Transport of D₂O into the system can be prevented or limited by means well known in the prior art. Examples of these means are inter alia targeted manipulation of the osmotic gradient across the skin (i.e. between systemic part and the skin surface) by reducing the water potential of the topically applied D₂O by means of substances suitable for altering this water potential, in particular physiologically tolerated salts such as sodium chloride, water-soluble polymers and other non-pharmaceutical substances. An organism to be treated in the context of the present invention refers especially to an animal organism, especially a mammal, a human or non-human mammal such as, for example, human, rat, mouse, horse, pig, sheep and goat.

Thus, a healing effect without systemic or other severe side effects has been detected according to the invention after topical application of D₂O as active ingredient to a human skin suffering from psoriasis. It has further been possible to show in the case of scars of the skin, especially hypertrophic scars and keloids, a reduction in the hypertrophy of a scar through the topical application of D₂O. It has also been possible to detect through the application of D₂O in skin cultures a retardation or inhibition of the growth of malignant and non-malignant hyperproliferative cells. Finally, it has been possible to treat successfully keratoses and in this connection especially acne and diabetic foot by topical application of D₂O.

A preferred embodiment therefore relates to the use according to the invention of D₂O by topical application of D₂O to the skin, preferably to the skin of an animal organism, especially of a mammal, of a human or non-human mammal, such as, for example, human, rat, mouse, horse, pig, sheep and goat. The topical application preferably takes place by
1) direct application as liquid or formulation, in particular ointment, cream or gel (described in detail hereinafter);
2) application via a patch or a bandage (described in detail hereinafter) or
3) application as aerosol (described in detail hereinafter).

A further preferred embodiment relates to the use according to the invention of D₂O, where D₂O retards and/or inhibits the proliferation of skin cells.

“Skin cells” means in the context of the present invention all proliferating cells of the skin, for example, but not restricted thereto, keratinocytes, epidermal cells, dermal cells, fibroblasts, collagen cells, connective tissue cells and melanocytes.

The term “topical” or “topical application” or “topical administration” or “topical use” in the context of the present invention means the local application or introduction of active ingredients, e.g. pharmaceutical active ingredients or non-pharmaceutical active ingredients, onto the skin, preferably as liquid, gas or formulation, in particular ointment, cream or gel. In contrast to systemic administration of active ingredients, uptake of the active ingredients takes place without transport to the site of action via the blood stream and therefore ordinarily has fewer side effects than systemic administration, because high active ingredient concentrations are present only in the locally treated region of the skin.

The term “retard” or “retardation” in the present connection means that the proliferation rate of hyperproliferative cells is slowed and/or reduced, preferably by up to about 5%, with preference by up to about 10%, likewise preferably by up to about 20%, more preferably by up to about 30%, likewise more preferably up to about 40%, even more preferably up to about 50%, most preferably up to about 60%, compared with the proliferation rate of these hyperproliferative cells without administration of D₂O. The term “inhibit” or “inhibition” in the present connection means that the proliferation rate of hyperproliferative cells is slowed or reduced preferably more than 50%, likewise preferably up to about 60%, further preferably up to about 65%, with preference up to about 70%, likewise preferably up to about 80%, more preferably up to about 90%, likewise more preferably up to about 95%, even more preferably up to about 98%, most preferably by up to 100%, compared with the proliferation rate of these hyperproliferative cells without administration of D₂O.

During the use of the D₂O-containing compositions of the invention, especially for the therapy of psoriasis, it has been observed that the effect is maintained for the whole observation period of 4 weeks after termination of the therapy. A preferred use of the invention therefore relates to the administration, especially the topical application, of D₂O, D₂O-containing compositions and D₂O in combination with one or more further active ingredients at intervals, i.e. with treatment-free periods. The treatment-free periods preferably amount to 1 week or more, preferably 2, 3, 4, 5, 6, 7, 8, 9, 10 or more weeks. The duration of treatment between the treatment-free periods may vary, preferably between 1 day and 50 days, in particular 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days. The first treatment is preferably carried out until the symptoms of the particular hyperproliferative disorder have substantially regressed. The treatment period necessary for this depends on the hyperproliferative disorder treated in each case. The physician treating in each case will decide whether the treatment has given the desired result. For example, in the case of psoriasis, the treatment period until the symptoms have substantially regressed is between 1 and 10 days, in the treatment of keratoses, especially acne and diabetic foot, it is between 10 and 50 days, and in the treatment of hypertrophic scars it is between 5 and 30 days.

In a particularly preferred embodiment of the therapy of psoriasis, D₂O, a D₂O-containing composition or D₂O in combination with one or more further active ingredients is applied for a period of from 1 to 10 days, e.g. 1, 2, 3, 4, 5, 6, 7, 8, 9 or 10 days, to the afflicted areas of skin. This treatment is repeated at intervals of from 1 to 10, e.g. 1, 2, 3, 4, 5, 6, 7, 8, 9, 10 weeks.

The present invention additionally shows not only the effect of D₂O alone in retarding or inhibiting the rate of division of hyperproliferative cells in diseased skin tissue, but also that administration of a combination of D₂O with another pharmaceutical active ingredient is able to enhance this effect. It is likewise possible, in addition to or instead of a further pharmaceutical active ingredient, to use D₂O together with a further non-pharmaceutical active ingredient, in particular for optimizing the topical application of D₂O as pharmaceutical active ingredient on the skin. Such a combination of D₂O and at least one further pharmaceutical active ingredient and/or at least one further non-pharmaceutical active ingredient is referred to hereinafter as “combination of the invention”.

Consistent therewith, all the uses and (topical) applications of D₂O according to the invention which are disclosed in this description can likewise be applied without restrictions to a combination of the invention unless the opposite is indicated. Likewise, uses of a combination of the invention in relation to the layered systems, mixtures of D₂O and H₂O, patches and bandages, formulations and aerosols of the invention (see below) are applied without restriction unless the opposite is indicated.

A preferred embodiment of the present invention accordingly relates to the use according to the invention of D₂O, where the D₂O is used together with at least one further pharmaceutical active ingredient and/or at least one further non-pharmaceutical active ingredient. The at least one further pharmaceutical active ingredient is preferably selected from the group consisting of cytostatics, proteins, peptides, nucleic acids, immunosuppressive active ingredients such as corticoids, and growth factors. The at least one further non-pharmaceutical active ingredient is preferably selected from the group consisting of pharmaceutically acceptable inorganic or organic acids or bases, polymers, copolymers, block copolymers, monosaccharides, polysaccharides, ionic and nonionic surfactants or lipids, and mixtures thereof. Also included in this group are specific proteins such as albumin, transferrin and kinase inhibitors. The aim of such combinations of D₂O and further pharmaceutical and/or non-pharmaceutical active ingredients is to enhance the antiproliferative effect and/or to improve the topical application of D₂O on the skin.

The term “active ingredient” used herein encompasses all pharmaceutical and non-pharmaceutical active ingredients of the present invention. The term “pharmaceutical active ingredient” is used herein as synonymous with the term “medicament” and refers in the context of the present invention to any inorganic or organic molecule, substance or compound having a pharmacological effect. The effect of a pharmaceutical active ingredient of the invention is an antiproliferative effect on cells of the skin, so that “antiproliferative pharmaceutical active ingredients” or else “antiproliferative active ingredients” are involved. D₂O is also to be regarded as a pharmaceutical active ingredient according to the present invention. The term “non-pharmaceutical active ingredient” refers in the context of the invention to any pharmacologically acceptable and therapeutically useful molecule, substance or compound which is not a pharmaceutical active ingredient but is administered preferably together with at least one pharmaceutical active ingredient to an organism to be treated, for example formulated in a formulation of the invention, in order to influence, in particular improve, qualitative properties of the pharmaceutical active ingredient(s) or of the formulation of the invention. The non-pharmaceutical active ingredients preferably display no pharmacological effect or one which is negligible or at least not undesired in relation to the intended therapy. Examples of suitable non-pharmaceutical active ingredients are pharmacologically acceptable salts, for example sodium chloride, flavorings, vitamins, e.g. vitamin A or vitamin E, tocopherols or similar vitamins or provitamins occurring in the human organism, antioxidants such as, for example, ascorbic acid, and stabilizers and/or preservatives to prolong the duration of use and storage of a pharmaceutical active ingredient or of a formulation of the invention, and other customary non-pharmaceutical active ingredients or excipients and additives known to the person skilled in the art. Further non-pharmaceutical active ingredients preferred according to the invention are in particular all substances able to form aqueous gels, such as, for example, natural or synthetic water-soluble polymers able to form networks.

Further pharmaceutical active ingredients preferred according to the invention for a combination of the invention and the effect thereof are detailed hereinafter, this list being only by way of example, and the present invention not being restricted thereto:

Water-Soluble Cytotoxic Acid Ingredients

An increase in the cytotoxic activity in the selected area of skin can be achieved by adding water-soluble cytotoxic active ingredients, especially, but not exclusively, cytostatics or mixtures of a plurality of cytostatics. The term “cytostatics” refers to natural or chemical substances or compounds which intervene in the cell cycle of proliferative cells by considerably delaying, retarding or inhibiting the proliferation, for example by retardation of DNA synthesis with short-term RNA and protein synthesis retardation. It is likewise possible to achieve a persistence of the D₂O effect in the sense of reducing the neoplastic regions in the skin even after termination of the D₂O application by adding any water-soluble cytostatics suitable for the treatment of hyperproliferative malignant skin disorders, such as, for example, but not restricted thereto, bleomycin, cyclophosphamide, doxorubicin, paclitaxel (Taxol), vincristine or mixtures of a plurality of such cytostatics. At the same time, the side effects on organs which are not to be treated, such as liver and kidney, are reduced, because D₂O is applied in directed fashion to the skin and reaches the system only in low concentrations. Systemic spread of D₂O and afore-mentioned active ingredient(s) is thus almost completely prevented.

Examples of cytostatics suitable according to the invention are (classified according to their mechanism of action):
alkylating and crosslinking cytostatics (which damage DNA): cyclophosphamide, N-nitroso compounds such as carmustine, ethyleneimine (aziridine) derivatives such as thiotepa, methanesulfates such as busulfan, platinum complexes such as cisplatin, and procarbazine,
cytostatic antibiotics: anthracyclines such as daunorubicin, doxorubicin, bleomycin and mitomycins (the latter intercalate into DNA and retard topical isomerases), and
antimetabolites (they displace natural metabolic components): folic acid antagonists such as methotrexate, and nucleoside analogs such as mercaptopurine, fluorouracil.

The specific activity can be further enhanced by application of cytostatics which are conjugated to other, preferably pharmaceutically active, molecules (for example polypeptides). Further examples of the enhancement of the specific activity of cytostatics are substances and pharmaceutical active ingredients from the group of antibiotics and of chemotherapeutics.

Immunosuppressive Active Ingredients

The response of the skin tissue to D₂O treatment, especially when inflammations are already present, can be improved and optimized by adding corticoids and/or other immunosuppressive active ingredients or immuno-modulators.

Growth Factors

The differential effect of D₂O on hyperproliferating cells can be further enhanced by adding growth factors such as, for example, but not restricted thereto, TNF-alpha, TGF-beta, PDGF, IGF, interleukin-4, endothelin-1, CTGF and VEGF. Whereas D₂O preferentially enters the hyperproliferating regions of the skin tissue and there retards or inhibits cell division, the growth factors are distributed homogeneously over normal and hyperproliferative regions. In the normal regions they may stimulate the growth of these cells or tissues, whereas their effect in the hyperproliferative regions is substantially inhibited through the presence of higher D₂O concentrations. It is thus possible to implement a differential therapeutic strategy in which the growth of healthy tissues is promoted and the growth of diseased cells or tissues is inhibited.

Nucleic Acids

It is possible by adding nucleic acids, in parallel with the cytotoxic effect of D₂O, to achieve an alteration in the genetic information of the cells in the region of the site of action or a targeted switching off (“gene silencing”) of particular genes of cells in the region of the site of action within the skin tissue and thus a modification of the proteome. The “gene silencing” may lead for example to the genes involved in defense against DNA damage (for example p53, BRCA1, BRCA2, ATM, CHK2) being switched off and thus the hyperproliferative cells whose cell division is impeded by D₂O no longer proliferating even in the long term (after the end of topical D₂O administration), but being eliminated finally be apoptosis. Methods for carrying out a “gene silencing” are well known to a person skilled in the art and are described for example in Mello C C, Conte D “Revealing the world of RNA interference”, in Nature 431, 338-342 (Sep. 16, 2004). The nucleic acids are preferably DNA, preferably oligonucleotides, sense or antisense DNA, natural or synthetic, cDNA, genomic DNA, naked DNA, single- or double-stranded DNA, or circular DNA, or RNA, preferably antisense RNA, RNAi, siRNA, or other RNA molecules suitable for interference, the length of which is not restricted.

The concentration of further pharmaceutical active ingredients used according to the invention in addition to D₂O as pharmaceutical active ingredient, based on the complete solution of a combination of the invention, is in the region of at least 10⁻⁸ M to at least 5×10⁻² M, preferably of at least 10⁻⁷ M to 10⁻³ M, most preferably of at least 10⁻⁶ M to at least 10⁻² M. A particularly preferred concentration range is in the region of at least 10⁻⁹ M to at least 10⁻² M.

In a particularly preferred embodiment, the active ingredient combinations of the invention include D₂O and vitamin D derivatives which act as agonists of the vitamin D receptor, especially calcipotriol; retinoid derivatives which act as agonists of the retinoid receptor (RAR), especially tazarotene; corticosteroid derivatives which act as agonists of the glucocorticoid receptor, especially betamethasone and cortisone; fumaric acid, skin-thinning agents, especially clobetasol; antagonists of dihydrofolate dehydrogenase, for example metothrexate and immunosuppressive substances, for example amphotericin, busulphan, cotrimoxazole, chlorambucil, colony stimulating factor, cyclophosphamide, fluconazole, ganciclovir, methylprednisolone, octreotide, oxpentifylline, riluzol, thalidomide, zolimomab aritox. and calcineurin antagonists, especially cyclosporins, e.g. cyclosporin A, cyclosporin G, cyclosporin B, cyclosporin C, cyclosporin D, dihydro-cyclosporin D, cyclosporin E, cyclosporin F, cyclosporin H, cyclosporin I, pimecrolimus, or tacrolimus.

Further non-pharmaceutical active ingredients which are preferred according to the invention in a combination of the invention and the effect thereof, and suitable concentrations, are detailed hereinafter, this list being only by way of example, and the present invention not being restricted thereto:

Water-Soluble Excipients and Additives

The physiological tolerability of D₂O on and in the skin for normally proliferating cells can be improved by adding water-soluble excipients and additives such as, for example, pharmaceutically acceptable inorganic or organic acids, bases, salts and/or buffer substances to adjust the pH.

Examples of preferred inorganic acids are selected from the group consisting of hydrochloric acid, hydrobromic acid, nitric acid, sulfuric acid and phosphoric acid, with particular preference for hydrochloric acid and sulfuric acid. Examples of particularly suitable organic acids are selected from the group consisting of malic acid, tartaric acid, maleic acid, succinic acid, acetic acid, formic acid and propionic acid and particularly preferably ascorbic acid, fumaric acid and citric acid. It is also possible where appropriate to employ mixtures of said acids, especially of acids which, besides their acidifying properties, also have other properties, e.g. in use as flavorings or antioxidants, such as, for example, citric acid or ascorbic acid. Examples of pharmaceutically acceptable bases are alkali metal hydroxides, alkali metal carbonates and alkali metal ions, preferably sodium. Mixtures of these substances can be used in particular for adjusting and buffering the pH, with particular preference in this connection for potassium hydrogenphosphate and dipotassium hydrogenphosphate, and sodium hydrogenphosphate and disodium hydrogenphosphate. Preferred buffer substances in the context of the invention are in addition PBS, HEPES, TRIS, MOPS and further physiologically tolerated substances with a pK in the range from 5.0 to 9.0. The concentration of these substances, based on the complete solution of a combination of the invention, is preferably in the micromolar to millimolar range, particularly preferably in the 1-100 mM range.

Water-Soluble, Non-Cytotoxic Molecules

An additional delay (retardation) of the transfer of the D₂O from topical application form to the skin, and from the skin into the system, can be achieved by adding water-soluble, non-cytotoxic molecules such as, for example, certain polymers (e.g., but not restricted thereto, dextran, polyethylene glycol, agarose, cellulose, hylaronic acid), copolymers and block copolymers, through the high water-binding capacity thereof. It is additionally possible through the ability of the polymers to reduce the chemical potential (water potential) of D₂O to alter and improve and optimize the strength and direction of the osmotic gradient across the skin. The concentration of these substances, based on the complete solution, is in the micromolar to molar range, preferably in the 1-500 mM range.

Water-Soluble, Non-Polymeric Molecules

It is possible by adding water-soluble, non-polymeric molecules which alter the density and/or viscosity of D₂O, for example, but not restricted thereto, monosaccharides and polysaccarides, especially glucose, sucrose, dextrose, maltose, starch and cellulose, to alter and optimize the osmotic conditions in the region of the topical D₂O application, and the D₂O transport and D₂O retention in the skin. The concentration of these substances, based on the complete solution of a combination of the invention, is preferably in the millimolar to molar range, particularly preferably in the range from 1.0 mM to 1.5 M.

Substances Altering the Interfacial Tension of D₂O

It is possible by adding substances which alter the interfacial tension of D₂O, for example, but not restricted thereto, ionic and nonionic surfactants or lipids, especially a mixture of surfactants and lipids, to alter the transport of the D₂O from the topical application into the skin and within the skin. In addition, molecules of these types are able in combination with the water-soluble cytotoxic active ingredients mentioned above in relation to the pharmaceutical active ingredients, especially cytostatics, to assume the role of a solubilizer for cytotoxic active ingredients, especially cytostatics, which are slightly soluble in D₂O, and thus inter alia, but not restricted thereto, to make it possible for larger amounts of the cytotoxic active ingredient, especially cytostatic, to be transported per dose, in particular per topical application (concerning topical applications according to the invention, see hereinafter). The concentration of these substances, based on the complete solution of a combination of the invention, is preferably in the micromolar to millimolar range, particularly preferably in the 1-500 mM range.

Water-Soluble Molecules which are Preferentially Taken Up by Strongly Proliferating (Hyperproliferative) Cells

It is possible by adding water-soluble molecules which are known to be taken up to a particular extent by strongly proliferating cells, for example albumin or transferrin, to achieve an additional increase in the D₂O transport rate of the molecules surrounded by a D₂O hydration sheath into the target cells of the skin.

DNA Repair Molecules

It is possible by adding molecules suitable for preventing the repair and restoration of damaged DNA strands (especially, but not exclusively DNA repair proteins such as kinase inhibitors) and/or inducing the apoptosis of cells with damaged DNA, to achieve a persistence of the D₂O effect even after the end of the administration of D₂O.

The concentration or dosage of D₂O and, where appropriate, of the at least one further pharmaceutical and/or non-pharmaceutical active ingredient is subject to various factors, for example the mode of treatment, type of disorder, pathological condition of the patent (mammal), nature of the active ingredient etc. Such parameters are known to a person skilled in the art, and determination of the specific dosages is subject to the expert knowledge of the person skilled in the art. Suitable concentration data are disclosed herein. Some examples of data on suitable concentration ranges have been detailed above, but these are intended to represent only guideline values.

The D₂O which can be used according to the invention is preferably in the form of a liquid. The D₂O is preferably in the form of a solution, preferably H₂O (water) as solvent, and is also referred to herein as “mixture of D₂O and H₂O” if H₂O is present, or as “D₂O solution” or “pure D₂O” if no H₂O is present. A mixture of D₂O and H₂O according to the invention comprises D₂O preferably in a concentration range from 1 to 99%, preferably from 5 to 98%, further preferably from 10 to 90%, likewise preferably from 15 to 80%, more preferably from 20 to 70%, likewise more preferably from 30 to 60%, most preferably from 40 to 50%, where these data relate to the total water content of the mixture of D₂O and H₂O. A D₂O solution of the invention and, in analogy thereto, a combination of the invention are prepared for example by mixing the components, in particular D₂O and, if appropriate, H₂O, and where appropriate the at least one further pharmaceutical and/or non-pharmaceutical active ingredient. A further possibility where appropriate is to add a solvent, as described hereinafter, by mixing. The admixture of H₂O and of the at least one further pharmaceutical and/or non-pharmaceutical active ingredient and of the solvent to D₂O preferably takes place in the liquid state of aggregation. However, preparation can also be achieved by any suitable process. In the event that D₂O is used alone (i.e. not in combination with further pharmaceutical and/or non-pharmaceutical active ingredients) and at a concentration of 100% based on the total volume of the solution (i.e. pure D₂O), pure D₂O represents the D₂O solution of the invention.

All the uses and (topical) applications of D₂O according to the invention disclosed in this description can also be applied without restriction to a mixture of D₂O and H₂O according to the invention unless the opposite is indicated. Likewise, uses of a mixture of D₂O and H₂O of the invention in relation to the combinations, layered systems, patch and bandage, formulations and aerosols (see below) of the invention apply without restriction, unless the opposite is indicated.

The use of D₂O according to the invention can, as described in detail hereinafter, likewise take place as aerosol, vapor or formulation, in particular as cream, ointment or gel. The concentrations of D₂O to be used in this connection are likewise dealt with hereinafter.

In a preferred embodiment, the at least one further pharmaceutical active ingredient or further non-pharmaceutical active ingredient is bound to D₂O. “Bound” in the context of the present invention means that the pharmaceutical and/or non-pharmaceutical active ingredient is hydrated by the D₂O.

In further preferred embodiments, the D₂O or the combination of the invention is present in a suitable solvent. A solvent of the invention may be an inorganic or organic solvent. Suitable solvents of the present invention ought preferably to be physiologically well tolerated by the organism (in particular mammal) to which the active ingredient with solvent is administered, i.e. not induce any side effects, e.g. toxic side effects. A particularly preferred solvent is distilled water. Ethanol-water mixtures are likewise preferred; in this case, the percentage content of ethanol by mass in these mixtures is preferably in the range between 5% and 99% ethanol, likewise preferably in the range from 10% to 96% ethanol, more preferably between 50% and 92%, most preferably between 69% and 91% ethanol.

D₂O or a combination of the invention may be in “preformulated” form, for example packed in suitable means for transporting pharmaceutical active ingredients, so-called drug delivery systems, for example in nanoparticles, vectors, preferably gene transfer vectors, viral or nonviral vectors, poly- or lipoplex vectors, liposomes or hollow colloids (i.e. hollow spheres of colloidal dimension). Also suitable for transport are naked nucleic acids, in particular naked DNA. Suitable vectors, liposomes, hollow colloids or nanoparticles, and processes for introducing substances into such vectors, liposomes, hollow colloids or nanoparticles are generally well known in the prior art and described for example in Cryan S-A., Carrier-based strategies for Targeting Protein and Peptide Drugs to the Lungs, AAPS Journal, 2005, 07(01):E20-E41 and Sambrook et al. Molecular Cloning. A Laboratory Manual, Cold Spring Harbor Laboratory (1989) NY. Gene transfer vectors which can be used are preferably polyethyleneimines or cationic lipids such as, for example, DOTAP. Liposomes can preferably be used for the packaging of cytostatics; a detailed description takes place for example in Koshkina N V et al., Koshkina, N. V., et al., Paclitacel liposome aerosol treatment induces inhibition of pulmonary metastases in murine renal carcinoma model., Clinical Cancer Research, 2001, 7, 3258-3262. Proteins as pharmaceutical active ingredients can preferably be packaged by means of supercritical fluids, emulsion processes and spray drying into biocompatible polylactic/glycolic acid polymers (PLGA).

Topical application of D₂O is likewise possible via a patch or a bandage. A further preferred embodiment accordingly relates to the use of D₂O according to the invention, where D₂O is topically applied with or via a patch or a bandage. The D₂O can preferably be used with at least one further pharmaceutical active ingredient and/or at least one further non-pharmaceutical active ingredient according to the present invention. The at least one further pharmaceutical active ingredient is preferably selected from the group consisting of cytostatics, proteins, peptides, nucleic acids, immunosuppressive active ingredients such as corticoids, and growth factors. The at least one further non-pharmaceutical active ingredient is preferably selected from the group consisting of pharmaceutically acceptable inorganic or organic acids or bases, polymers, copolymers, block copolymers, monosaccharides, polysaccharides, ionic and nonionic surfactants or lipids, and mixtures thereof. Also included in this group are specific proteins such as albumin, transferrin and kinase inhibitors.

“Patches” or “bandages” in the context of the invention mean all devices which can be fixed on the skin by mechanical or chemical interaction, physisorption, adhesion or other physicochemical processes and which are suitable for covering a selected region of skin occlusively or non-occlusively over a period sufficiently long for the intended treatment and for making possible and/or assisting the delivery of D₂O to the skin. Patches and bandages which can be used according to the invention as application systems for local release of active ingredients to the skin (e.g. heat patches) and for controlled systemic release of active ingredients (e.g. opiate depot patches, nitroglycerin depot patches) are known in the prior art. “Depot patch” or “depot bandage” is intended to mean, in addition to the properties described above, the ability of the patch or the bandage to store D₂O and the controlled delivery thereof to the skin over a period of days or weeks. Such depot patches and depot bandages are encompassed hereinafter by the terms patch and bandage, respectively.

A preferred embodiment of the present invention is accordingly a patch or bandage for topical application comprising D₂O, Such a patch or bandage of the invention can preferably further comprise at least one further pharmaceutical active ingredient and/or at least one further non-pharmaceutical active ingredient. The at least one further pharmaceutical active ingredient is preferably selected from the group consisting of cytostatics, proteins, peptides, nucleic acids, immunosuppressive active ingredients, such as corticoids, and growth factors. The at least one further non-pharmaceutical active ingredient is preferably selected from the group consisting of pharmaceutically acceptable inorganic or organic acids or bases, polymers, copolymers, block copolymers, monosaccharides, polysaccharides, ionic and nonionic surfactants or lipids, and mixtures thereof. Also included in this group are specific proteins such as albumin, transferrin and kinase inhibitors.

A patch of the invention or a bandage of the invention can likewise preferably comprise a mixture of D₂O and H₂O, Such a patch or bandage may also preferably further comprise at least one further pharmaceutical and/or at least one further non-pharmaceutical active ingredient of the invention, as detailed above.

A patch of the invention or a bandage of the invention is used for the topical application of D₂O to the skin. The patch or the bandage comprises D₂O preferably in an arrangement which stores the D₂O in the form of a depot and makes controlled delivery thereof to the skin possible.

Overall, the following problem must be taken into account for every controlled release of D₂O to the skin: the release from a liquid or formulation as ointments, cream or gel applied directly to the skin may be impeded by the direction of the osmotic gradient within the skin from the inside to the outside because the D₂O has in the liquid, ointment, cream or gel in some circumstances a lower water potential than the H₂O in the skin and the underlying vessels.

A particularly preferred embodiment of the topical application of D₂O is therefore to regulate and therefore to control the depth or degree of penetration of D₂O into the skin by targeted manipulation of the osmotic conditions in the region of skin to be treated, specifically and preferably to the depth at which the hyperproliferating cells are located, preferably as far as the epidermis or as far as the dermis. This can be achieved by the chosen composition of an applied combination of the invention, by adding substances able to alter the osmotic conditions on the surface of the skin. Examples of such substances are detailed hereinbefore.

A further possibility for the controlled penetration of D₂O into the skin consists of using one or more membrane(s) or film(s) which make(s) it possible for water and gases to pass through but prevent(s) larger molecules or particles (including bacteria, viruses, protozoa). Examples of such membranes and films which can be used according to the invention are known in the prior art and have numerous applications such as, for example, in textiles under the brand name GORE Tex® or in medicine so-called biofilms.

A very particularly preferred embodiment of the invention therefore consists of the use according to the invention of a patch or a bandage, where the patch or the bandage is used in combination with at least one membrane or at least one film. The at least one membrane or the at least one film is preferably a micro- or nanoporous membrane or film.

The present invention likewise encompasses a membrane or film which already itself has the function of a patch or a bandage of the invention, and thus represents a device which can be fixed per se, preferably self-adhesively, for the occlusive or non-occlusive covering of defined regions of skin. Examples thereof are hypoallergenic nano- or microporous “tape strips” or plasters for covering wounds (e.g. Tegaderm®).

In a particularly preferred embodiment, D₂O is topically applied to the skin via a special arrangement. This arrangement consists of the following components:
a micro- or nanoporous membrane or film which is applied directly to the skin,
followed by a D₂O layer which comprises the D₂O,
where appropriate followed by a so-called occlusive layer which prevents or regulates evaporation of D₂O to the outside and, at the same time, represents a mechanical protection which prevents the escape of D₂O as liquid, and
a patch or a bandage.

In a further preferred embodiment, D₂O is topically applied to the skin via a special arrangement. This arrangement consists of the following components:
a previously described micro- or nanoporous membrane or film which already itself has the function of a patch or a bandage of the invention, which is applied directly to the skin,
followed by a D₂O layer which comprises the D₂O,
where appropriate followed by a so-called occlusive layer which prevents or regulates vaporization of the D₂O to the outside and, at the same time, represents a mechanical protection which prevents the escape of D₂O as liquid, and
a further micro- or nanoporous membrane or film which already itself has the function of a patch or a bandage of the invention.

If required, it is of course possible to add further D₂O layers which comprise further D₂O and which are separated by membranes or films, thus making it possible to alter the depot effect or the transfer of the D₂O into the skin, for example the amount and/or duration of release of D₂O. The term “D₂O layer” used relates to liquid pure D₂O, a mixture of D₂O and H₂O according to the invention and a formulation of D₂O in particular as cream, ointment or gel.

It is likewise preferably possible also to add layers which have chemical, electrical or thermal properties which are suitable for manipulating the transfer of the D₂O into the skin and/or the duration of the release thereof. Examples thereof are layers suitable for setting up and/or maintaining an electrical, thermoelectrical, thermal or chemical potential (or a combination thereof) across the underlying layers and the skin. This can be achieved for example by electrodes which are embedded in membranes or films of the invention, or present thereon, and which are supplied from outside with current (DC voltage, AC voltage or high-frequency currents) or which generate, through the specific choice of the electrode material, electrochemical potentials with the D₂O layer as electrolyte.

The totality of such previously described D₂O layers, occlusive layers, layers with chemical, electrical or thermal properties, membranes and films in any number, combination and arrangement suitable for the purpose of use is referred to according to the invention as “layered system”. Such a layered system is preferably used in conjunction with a previously described (depot) patch or (depot) bandage.

The transfer of the D₂O from a patch, bandage or layered system of the invention into the skin can be influenced or altered in a targeted manner by varying the morphology (pore size, membrane or film thickness, surface roughness and surface profile) and surface properties (e.g. hydrophilic or hydrophobic, chemical layers—covalently bonded or adhesively bonded—, functional groups, binding or incorporation of inorganic or organic substances) of the membrane or film which is in direct contact with the skin.

A further variation of the entry of D₂O into the skin is possible by the targeted use of adhesives which can be used for the mechanical fixing of the (depot) patch or the (depot) bandage to the skin but are not absolutely necessary. The adhesives generally used for topical applications of patches and bandages tend to have hydrophobic characteristics which can prevent the passage of D₂O through the adhesive layer. An alteration in these properties can be achieved by admixing additives to the adhesive preparation. Suitable “additives” of this type are organic and/or inorganic substances and compounds which are able to alter the permeation properties of D₂O through the adhesive layer. Examples of such substances are, inter alia, polymers, copolymers, block polymers, block copolymers, surfactants, peptides, proteins, nucleic acids, sterols and steroids.

In a further preferred embodiment, D₂O is topically applied to the skin, preferably with a patch or a bandage of the invention, via an arrangement which makes it possible for D₂O to be transferred into the skin substantially or exclusively via the vapor phase. This means according to the invention that D₂O used as liquid vaporizes as molecular D₂O and makes contact as vapor with the skin. The concentrations of the D₂O thus correspond to the concentrations described above of the (D₂O-containing) liquid of the invention. D₂O in vapor form has the advantage of particularly easy penetration into the skin. In order to bring about this vaporization, thermal energy is necessary, which can be provided either by the skin itself or by an external heat source, e.g. on use of a patch or a bandage of the present invention by electrical heating (e.g. Peletier heating) introduced into the layered system of the invention described above. In addition to this thermal energy it is possible by targeted modifications, for example the choice of the morphology (in particular pore size and surface coating), of a first membrane or film of the invention, which is present on the skin, of the layered system of the invention, to allow only D₂O in vapor form to penetrate as far as the skin, whereas liquid D₂O is retained. With this embodiment too it is possible by the alterations, described above, of the layered system of the invention and corresponding modifications to membranes or films which are further used where appropriate for the amount and the duration of the release of D₂O via the vapor phase to be controlled and/or altered.

A further preferred embodiment of the invention relates to the use according to the invention of D₂O, where the D₂O is applied as aerosol. The application preferably takes place directly onto the skin.

An “aerosol” means solid or liquid suspended particles with a diameter of about 0.0001 μm to about 100 μm, in gases, in particular air, where the composition and form of the aerosols may vary widely. The smallest pharmaceutically effective particles in aerosols are, for example, nucleic acids, peptides or proteins, and the largest particles are, for example, mist particles. Aerosols frequently consist of mixtures of particles of different particle sizes and thus incorporate a polydisperse size distribution. Aerosols can be produced artificially by dispersion and condensation processes well known in the prior art. They can be used without propellant gas or be used in conjunction with a liquefied compressed gas as propellant gas, for example in spray cans.

An aerosol of the invention is preferably applied via a nebulizer. “Nebulizer” means for the present invention any apparatus which is suitable for medical aerosols and with which aerosol particles in the size range from 50 nm to 50 μm can be produced. D₂O is supplied according to the invention to the nebulizer in order to produce therefrom aerosols, preferably propellant gas-free, of the invention. For this purpose, the nebulizer sprays a defined volume of the D₂O, usually with application of high pressures, through small nozzles in order thus to generate an aerosol of the invention which can be applied to the skin.

Suitable nebulizers for aerosols of the invention also include propellant gas-driven inhalers or nebulizers. Propellant gases may in this connection be for example CFC or HFC. Concerning this, reference is made to “Theorie und Praxis der Inhalationstherapie”, pages 31 to 70 Arcis Verlag (2000), where a detailed description of nebulizers which can be used and of methods for using them is/are disclosed.

Examples of nebulizers suitable according to the invention are compressed air-driven nozzle nebulizers (e.g. PARI LC plus, PARI GmbH, Starnberg, Germany), venturi nozzle nebulizers, water vapor-driven nozzle nebulizers or ultrasonic nebulizers (e.g. AeronebLab, Aerogen, Inc., Stierlin Court, Canada; eFLOW, PARI GmbH, Starnberg, Germany). Likewise suitable are nebulizers of a size such that they can be carried along by the patient (human), e.g. the Respimat@ as described in WO 97/12687. All the references cited therein are included in their entirety in the present invention.

A further aspect of the invention relates to an aerosol which comprises a mixture of D₂O and H₂O for application onto the skin.

The production of an aerosol of the invention and of a D₂O solution of the invention for application as aerosol can take place by means of suitable known standard techniques for aerosol production.

The concentrations of D₂O to be used in an aerosol of the invention depends on various factors, for example the purpose of use, pathological condition and are subject to the expert knowledge of a person skilled in the art. An aerosol of the invention comprises D₂O preferably in a concentration range of from 5 to 98% by weight, preferably from 10 to 90% by weight, likewise preferably from 15 to 80% by weight, more preferably from 20 to 70% by weight, likewise more preferably from 30 to 60% by weight, most preferably from 40 to 50% by weight.

The generated aerosols of the invention which comprise D₂O alone (pure D₂O), a mixture of D₂O or H₂O or a combination of the invention are applied directly to the skin in the region to be treated. This can take place for example through a chamber which can be placed on the skin and is open toward the latter and through which the aerosol is passed.

In a further preferred embodiment, the mixture of D₂O and H₂O of an aerosol of the invention and of the further pharmaceutical and/or non-pharmaceutical active ingredient of the invention, present where appropriate, is present in a solvent, preferably present in at least one inorganic or organic solvent. The solvent is preferably selected from the group consisting of ethanol, water and glycerol, and mixtures thereof.

All the uses and (topical) applications of D₂O according to the invention which are disclosed in this description are likewise applied without restriction to an aerosol of the invention, unless the opposite is indicated. Likewise, uses of an aerosol of the invention are applied without restriction in relation to a combination of the invention, mixture of D₂O and H₂O of the invention, and a formulation of the invention, unless the opposite is indicated and as long as the described use is suitable for a use of the components in the gaseous state (as aerosol).

A further preferred embodiment relates to the use of D₂O according to the invention, where D₂O is topically applied as formulation.

A further aspect of the present invention relates to a formulation for topical application to skin comprising D₂O. The formulation is preferably an ointment, cream or gel.

An “ointment” according to the present invention is a pharmaceutical preparation which is to be used externally and is composed of a base of spreadable substances, such as petrolatum, to which the actual pharmaceutical and/or non-pharmaceutical active ingredients are added, for example by mixing.

A “cream” in the context of the present invention means an ointment of the invention which may additionally comprise further ingredients such as cosmetic active ingredients, e.g. fragrances, colorants and/or emulsifiers, e.g. lecithin. A cream is generally distinguished from a lotion, this distinction usually being made as a function of the degree of the viscosity. However, a cream also means according to the invention a lotion.

A “gel” of the present invention is the solution of a macromolecular substance, e.g. agarose or acrylamide, whose concentration is so high that the dissolved macromolecules link up to give a sponge-like, three-dimensional structure in whose cavities a liquid is present. Gels thus have a relatively solid consistency. The viscosity is between liquid and solid. Such a liquid is preferably pure D₂O or a mixture of D₂O and H₂O according to the invention.

In a preferred embodiment, the formulation of the invention comprises, where the formulation at least one further pharmaceutical active ingredient and/or at least one further non-pharmaceutical active ingredient. The at least one further pharmaceutical active ingredient is preferably selected from the group consisting of cytostatics, proteins, peptides, nucleic acids, immunosuppressive active ingredients such as corticoids, and growth factors. The at least one further non-pharmaceutical active ingredient is preferably selected from the group consisting of pharmaceutically acceptable inorganic or organic acids or bases, polymers, copolymers, block copolymers, monosaccharides, polysaccharides, ionic and nonionic surfactants or lipids, and mixtures thereof, albumin, transferrin and DNA repair proteins, such as kinase inhibitors.

The production of a formulation of the invention, especially of an ointment, cream or gel, is described by way of example in the examples. If such a formulation comprises further pharmaceutical and/or non-pharmaceutical active ingredients, these are preferably added to the formulation by mixing. It can, however, take place by any standard process known in the prior art. Such processes are known to a person skilled in the art, likewise the concentrations to be selected for the components and substances to be used.

The concentrations of D₂O in a formulation of the invention are preferably in the following ranges:
for a cream or ointment preferably in the range from 2 to 90% by weight, preferably from 5 to 85% by weight, likewise preferably from 10 to 80% by weight, particularly preferably from 15 to 70% by weight, more preferably from 20 to 60% by weight and most preferably from 25 to 50% by weight and
for a gel preferably 5 to 99% by weight, preferably from 10 to 90% by weight, likewise preferably from 15 to 80% by weight, particularly preferably from 20 to 70% by weight, more preferably from 30 to 70% by weight and most preferably from 35 to 65% by weight.
   A person skilled in the art will choose the suitable concentration depending in particular on the present indication, the patient's condition, the severity of the disorder etc.

In a particularly preferred embodiment, a formulation of the invention further comprises at least one inorganic or organic solvent. The solvent is preferably selected from the group consisting of ethanol, water and glycerol, and mixtures thereof.

All the uses and (topical) applications of D₂O according to the invention disclosed in this description likewise apply to the formulations of the invention without restriction, unless the opposite is indicated. Likewise, uses of a formulation of the invention apply without restriction in relation to the layered systems, mixture of D₂O and H₂O, patches and bandages, combinations and aerosols of the invention, unless the opposite is indicated.

The formulation of the invention preferably further comprises at least one inorganic or organic solvent, preferably selected from the group consisting of ethanol, water and glycerol, and mixtures thereof.

The formulations of the invention are preferably administered topically. The production of a formulation comprising D₂O or a combination of the invention, and the production of an aerosol of the invention comprising a formulation, can take place in analogy to the procedures as described above for the production of a D₂O solution, a combination of the invention or an aerosol of the invention. The choice and concentration of the excipients and additives which are included where appropriate depends on the purpose of use of the formulation and is subject to the expert knowledge of a person skilled in the art. A formulation of the invention can be prepared as liquid, ointment, cream or gel. The production of such liquids, ointments, creams or gels can take place in analogy to the above description. Processes therefor are known in the prior art. Concentrations of the individual components or substances depend on the particular purpose of use, pathological condition etc. and are subject to the expert knowledge of the person skilled in the art.

Further particularly preferred embodiments of the present invention relate to the use and application according to the invention of D₂O as aerosol of the invention or as formulation of the invention together with at least one further pharmaceutical active ingredient and/or at least one further non-pharmaceutical active ingredient. Likewise particularly preferred embodiments of the present invention relate to an aerosol of the invention and to a formulation of the invention which comprises at least one further pharmaceutical active ingredient and/or at least one further non-pharmaceutical active ingredient, as described herein. The at least one further pharmaceutical active ingredient is preferably selected from the group consisting of cytostatics, proteins, peptides, nucleic acids, immunosuppressive active ingredients such as corticoids, and growth factors. The at least one further non-pharmaceutical active ingredient is preferably selected from the group consisting of pharmaceutically acceptable inorganic or organic acids or bases, polymers, copolymers, block copolymers, monosaccharides, polysaccharides, ionic and nonionic surfactants or lipids, and mixtures thereof. Also included in this group are specific proteins such as albumin, transferrin and kinase inhibitors.

The present invention is explained further below by means of examples, the latter not restricting the subject matters of the invention.

### DESCRIPTION OF THE FIGURES

FIG. 1:

Average number of papules and papulopustules in a square region of skin 10×10 cm in size on the upper dorsal area of test subjects with moderately severe acne vulgaris as a function of the treatment time (trial days) with a D₂O hydrogel (circular symbols) and with an H₂O hydrogel (square symbols). The counting of the inflamed follicles took place every 5 days from the start of the trial, and the trial extended over a total of 40 days. The site evaluated on each test subject was always the same (water-resistant marking on the skin). Each test subject received both the D₂O hydrogel and the H₂O hydrogel applied to areas which were sufficiently spatially separated and had a water-resistant marking on the upper dorsal area which included the areas used for counting.

FIG. 2:

Average number of comedones in a square region of skin 10×10 cm in size on the upper dorsal area of 32 test subjects with moderately severe acne vulgaris as a function of the treatment time (trial days) with a D₂O hydrogel (circular symbols) and with an H₂O hydrogel (square symbols). The counting of the non-inflamed follicles took place every 5 days from the start of the trial, and the trial extended over a total of 40 days. The site evaluated on each test subject was always the same (water-resistant marking on the skin). Each test subject received both the D₂O hydrogel and the H₂O hydrogel applied to areas which were sufficiently spatially separated and had a water-resistant marking on the upper dorsal area which included the areas used for counting.

FIG. 3:

Average number of papules and papulopustules in a square region of skin 10×10 cm in size on the upper dorsal area of test subjects with moderately severe acne vulgaris as a function of the treatment time (trial days) with a D₂O cream (circular symbols) and with an H₂O cream (square symbols). The counting of the inflamed follicles took place every 5 days from the start of the trial, and the trial extended over a total of 40 days. The site evaluated on each test subject was always the same (water-resistant marking on the skin). Each test subject received both the D₂O cream and the H₂O cream applied to areas which were sufficiently spatially separated and had a water-resistant marking on the upper dorsal area which included the areas used for counting.

FIG. 4:

Average number of comedones in a square region of skin 10×10 cm in size on the upper dorsal area of 14 test subjects with moderately severe acne vulgaris as a function of the treatment time (trial days) with a D₂O cream (circular symbols) and with an H₂O cream (square symbols). The counting of the non-inflamed follicles took place every 5 days from the start of the trial, and the trial extended over a total of 40 days. The site evaluated on each test subject was always the same (water-resistant marking on the skin). Each test subject received both the D₂O cream and the H₂O cream applied to areas which were sufficiently spatially separated and had a water-resistant marking on the upper dorsal area which included the areas used for counting.

FIG. 5:

Average number of papules and papulopustules in a square region of skin 10×10 cm in size on the upper dorsal area of test subjects with moderately severe acne vulgaris as a function of the treatment time (trial days) with a D₂O agarose hydrogel film (circular symbols) and with an H₂O agarose hydrogel film (square symbols) which was directly applied to the skin and changed every 5 days. The counting of the inflamed follicles took place every 5 days from the start of the trial, and the trial extended over a total of 40 days. The site evaluated on each test subject was always the same (water-resistant marking on the skin). Each test subject received both the D₂O agarose hydrogel film and the D₂O agarose hydrogel film applied to areas which were sufficiently spatially separated and had a water-resistant marking on the upper dorsal area which were identical to the areas used for counting.

FIG. 6:

Average number of comedones in a square region of skin 10×10 cm in size on the upper dorsal area of 10 test subjects with moderately severe acne vulgaris as a function of the treatment time (trial days) with a D₂O agarose hydrogel film (circular symbols) and with an H₂O agarose hydrogel film (square symbols) which was directly applied to the skin and changed every 5 days. The counting of the non-inflamed follicles took place every 5 days from the start of the trial, and the trial extended over a total of 40 days. The site evaluated on each test subject was always the same (water-resistant marking on the skin). Each test subject received both the D₂O agarose hydrogel film and the D₂O agarose hydrogel film applied to areas which were sufficiently spatially separated and had a water-resistant marking on the upper dorsal area which were identical to the areas used for counting.

### EXAMPLES

### Example 1

### Production of Hydrous Gels

The production of hydrous gels took place under sterile conditions. 3.0% by weight agarose was dissolved in pure D₂O, in pure H₂O or in a mixture of D₂O and H₂O, and the solution was then heated to 90° C. The D₂O used, from Sigma-Aldrich (Munich), had an isotopic purity of 98.5%. The hot solution was poured into Petri dishes to a height of 3.0 mm and cooled. The D₂O agarose gels, H₂O agarose gels or D₂O/H₂O agarose gels obtained in this way were stored under sterile conditions at 4° C. Alternatively, acrylamide gels (5% acrylamide) were produced under sterile conditions, with pure D₂O, pure H₂O or a mixture of D₂O and H₂O being degassed before adding the acrylamide (with 2.4% bisacrylamide) and heated to 40° C. After addition of acrylamide and bisacrylamide, the solution was mixed (vortex mixer, 1 min at 200 rpm) and then the catalysts tetramethylethylenediamine (TEMES; 1.0%) and ammonium persulfate (AP; 0.1%) were added, followed by mixing for 10 sec. The gels were then poured into Petri dishes (height of the gel 3 mm) and a layer of butanol was placed on top before hardening, in order to achieve a particularly smooth gel surface. For hardening, the gels were stored at 40° C. for 2 hours.

For the respective gels (agarose and acrylamide), if required 15% by volume propylene glycol was added to the pure D₂O, pure H₂O or the mixture of D₂O and H₂O before adding the gel former, in order to suppress microbial contamination on application of the gel.

### Example 2

### Production of an Occlusive Depot Patch with D₂O or H₂O as Active Ingredient

Commercially available Tegaderm plasters from 3M (Neuss, Germany) with a size of 6×7 cm were used for the preparation. The cardboard frame on the dystal side was removed from a first plaster and a D₂O— or H₂O-based agarose gel or acrylamide gel (diameter 2.5 cm, thickness 3 mm) produced as in Example 1 was then applied to the center of this side in 6 separate arrangements.

The 6 arrangements were as follows:

arrangement 1: agarose gel+D₂O
arrangement 2: agarose gel+H₂O
arrangement 3: agarose gel+1:1 mixture of D₂O and H₂O
arrangement 4: acrylamide gel+D₂O
arrangement 5: acrylamide gel+H₂O
arrangement 6: acrylamide gel+1:1 mixture of D₂O and H₂O

A layer of Parafilm (diameter 3 cm) cut in the shape of a circle was then placed from above onto the center of the gel, and a second Tegaderm plaster, from which the protective film on the adhesive side (proximal side) had previously been removed, was placed in register with the adhesive side oriented toward the gel/Parafilm layered system on the dystal side of the first plaster and affixed. The finished depot patch was stored at 4° C. until used. All the preparations were carried out under sterile conditions.

### Example 3

### Generation of D₂O Aerosols

The aerosols were generated using exclusively prior art equipment. A Pari LC Plus universal nebulizer (PARI GmbH, 82319 Starnberg, Germany) was used in combination with a Pari universal compressor which generated 200 mg/min polydisperse aerosol with an average particle size (median mass diameter) of 2.5 μm for pure H₂O and D₂O and of 2.5-4.5 μm for H₂O or D₂O (operating pressure 2.0 bar, flow rate of compressor air was 6.0 l/min). The particle size was measured by dynamic light scattering in a flow cuvette. The aerosol was generated at a temperature of 37° C. by appropriate thermostating of the nebulizer in a waterbath thermostat.

### Example 4

### Production of a Hydrous D₂O Cream

A commercially available colloid base for cream production (Avicel CL611 from FHG Pharmaceuticals, Philadelphia, USA) was mixed either with D₂O, with H₂O or with a mixture of D₂O and H₂O (in each case buffered at pH 7.0 with 50 mM phosphate buffer). For this purpose, 2.0% by weight Avicel CL611 was added by means of a laboratory mixer (Lighnin-Mixer from Aldrich Chemical Co., Milwaukee, USA) at 500 rpm to the pure D₂O, pure H₂O or mixture of D₂O and H₂O which had been preheated to 70° C. and was mixed for 2 min. The speed of rotation was then raised to 1000 rpm, and mixing was continued for 10 min. Several creams which additionally comprised further ingredients, especially pharmaceutical active ingredients (such as corticoids), were likewise produced. For this purpose, these further ingredients were subsequently added, after the mixing of the above-mentioned respective components into the cream base, and mixed once more at 1000 rpm for 10 min. The result was a homogeneous cream with long-term stability.

### Example 5

### Influence of D₂O and H₂O on the Proliferation Rate of Human Skin Cell Lines in Culture

The influence of D₂O and H₂O on the proliferation rate of carcinoma cells and normal cells was investigated on cell cultures. For this purpose, human TE 354.T epithelial cell lines derived from the human basal-cell carcinoma (American Type Culture Collection (ATCC), Rockville, USA) were used. Normal human epithelial cell lines from the skin of the same individual (TE 353.sk from ATCC) were used as control. The cells were transferred into 96-well cell culture plates (10⁴ cells per well) and allowed to grow to subconfluence. The medium used for this purpose was Dulbecco's modified Eagles medium (DMEM) to which 10% heat-deactivated fetal calf serum (LGC Promochem, Wesel, Germany) was added. The medium was changed, for the first time 2 days after introduction of the cells and then every 48 hours. At the subconfluent stage of the cell culture of the two cell lines, 30% D₂O, based on the complete medium, was added to the medium. In a control group of both cell lines, the identical amount of H₂O was added instead of D₂O, Subsequently, a zero point measurement was carried out as baseline for determining the cell-doubling rate for both cell lines by means of light microscopic cell counting (cell count/area) using a haemocytometer. 48 hours after the addition of D₂O or H₂O, in each case 6 wells of sample and control were examined comparatively for cell proliferation under the light microscope. These examinations were continued thereafter every 24 hours for the period of a week. From these values, the average cell-doubling rate n was determined from the gradient of a graphical plot of log {cell count} vs. time in accordance with the equation n=3.32 (log Nₜ−log N₀). Here, Nₜ is the cell count measured after time t, and N₀ is the cell count determined at the time when D₂O or H₂O was added (zero point measurement). The average cell-doubling rate <n> was determined by averaging the results over in each case 6 wells per time point. The results are summarized in Table 1. A disproportionately large reduction in <n> for the carcinoma cells compared with normal cells is evident owing to the addition of D₂O.

TABLE 1

| | |
|---|---|
| Carcinoma cells | Normal cells |

| <n> D₂O | <n> H₂O | <n> D₂O | <n> H₂0 |
|---|---|---|---|
| 0.23 ± 0.02 | 0.92 ± 0.08 | 0.41 ± 0.04 | 0.65 ± 0.04 |

Influence of D₂O and H₂O on the average cell-doubling rate <n>D₂O and <n>H₂O of cell cultures of human TE 354.T epithelial cell lines derived from the human basal-cell carcinoma (carcinoma cells) and from normal skin epithelial cell lines (normal cells) of the same individual (TE 353.sk)

### Example 6

### Influence of D₂O and H₂O on the Development of Scars of the Skin

Acrylamide gels with pure D₂O and pure H₂O (control) were prepared as in Example 1, with the D₂O or H₂O containing 15% by volume propylene glycol. The thickness of the gels was 2 mm, and pieces 2×4 cm in size were cut out of the gel.

9 rabbits (New Zealand, white) were injured on the ear, in each case on the ventricular side, by incisions with a diameter of 6 mm, and the resulting wounds were treated with the above gels starting on the 16^{th} day after the wounding in 6 rabbits, specifically 3 rabbits with D₂O acrylamide gels, 3 rabbits with H₂O acrylamide gels, and 3 rabbits underwent healing without treatment. Of the 6 rabbits, 3 were treated with a D₂O acrylamide gel and 3 with an H₂O acrylamide gel. For this purpose, the appropriate gel was applied to the wound and fixed with a 1 mm-thick self-adhesive silicone film (Biodermis). The gels were changed every 2 days. On the 24^{th} day after the wounding, a 3D scar measurement was performed on all 9 rabbits by strip projection (ATOS-1 system from GOM, Bibertal bei Ulm, Germany) in which the scar area and hypertrophic scar volume were determined without contact. The results are compiled in Table 2. There proved to be a significantly smaller formation of the scar in terms of scar volume for the 3 rabbits treated with D₂O acrylamide gel.

TABLE 2

| | Scar area | Scar volume |
|---|---|---|
| | (mm²) | (mm³) |
| D₂O gel | 58 ± 7 | 83 ± 14 |
| H₂O gel | 72 ± 8 | 124 ± 16 |
| Control | 78 ± 8 | 158 ± 17 |

Scar area and hypertrophic scar volume on the 24^{th} day after injury for D₂O-based (D₂O gel) and H₂O-based (H₂O gel) acrylamide gels applied to the wound on the 16^{th} day after the injury, and for scars not further treated after the injury (control). The indicated values are averages over in each case 6 scars, and the standard deviation calculated for the average.

### Example 7

### Influence of D₂O and H₂O Depot Patches on Psoriasis

The occlusive depot patches described in Example 2 were applied to psoriatic areas with a size of 2-3 cm diameter on the human skin (arms and legs) of subjects and left there for 3 days. In total, on the same individuals in each case 5 sites on the skin were covered with D₂O patches and a further 5 with H₂O patches. A preparation based on an agarose gel according to Example 1 was used for in each case 3 of the D₂O and H₂O patches, and a preparation based on an acrylamide gel according to Example 1 was used for in each case 3 of the D₂O and H₂O patches. The selected areas were notable for intense itching, skin erythema and scaling. After removal of the patches, 72 hours after application thereof, the treated areas of skin were analyzed by dermascopy. In the case of the areas treated with D₂O patches, the treated skin was no longer visually distinguishable from the surrounding healthy skin, and itching, erythema and scaling had completely disappeared. No return of the symptoms was observable for an observation period of up to four weeks after removal of the patches. For the H₂O patches there was found to be a slight decline in itching, erythema and a disappearance of scaling, but all three symptoms returned again 2 days after removal of the patches and, after a further 2 days, were manifest entirely as before the treatment, i.e. present to the same extent as before the treatment. The results proved to be independent of the nature of the polymer used for the gels, i.e. no differences were observable for agarose or acrylamide gels.

### Example 8

### Influence of D₂O- and H₂O-Containing Cream Preparations on Psoriasis

Creams prepared as in Example 4 with either pure D₂O or pure H₂O were applied thinly every 6 hours to psoriatic skin areas 2-3 cm in size of human subjects' skin. The amount of cream put on per application corresponded to an amount of D₂O or H₂O of 60-70 μl per square centimeter of skin. The application was continued for 3 days and terminated after 72 hours. The treated areas of skin then underwent dermascopic analysis. For the D₂O-containing cream, the treated skin was no longer visually distinguishable from the surrounding healthy skin after 3 days, and itching, erythema and scaling had substantially disappeared. This effect persisted for the observation period of 4 weeks. For the H₂O-containing cream, a slight decline in erythema and a slightly reduced scaling and itching were found, but the symptoms returned after 3 days.

### Example 9

### Production of Hydrogels Based on Acrylic Acid

1.0% by weight Carbopol 980 (manufacturer: Noveon, Inc., 9911 Brecksville Rd., Cleveland, Ohio 44141-3247, USA) and 0.1% by weight sorbic acid were dissolved in separate mixtures by stirring in pure D₂O, in pure H₂O or in a mixture of D₂O and H₂O (1:1), and then titrated to a pH of 6.8 by pipeting 10 M NaOH solution. The colorless, transparent and optically clear acrylic acid gels (carbopol gels) (D₂O carbopol gel, H₂O carbopol gel, D₂O/H₂O carbopol gel) which had resulted through the addition of NaOH as a consequence of crosslinking of the polyacrylic acid via its carboxyl groups with the alkaline hydroxyl groups were then stored at room temperature until used further, for at least 24 hours. The D₂O from Sigma-Aldrich (Munich) used in this and all following examples had an isotopic purity of 99.0%.

### Example 10

### Production of Hydrogels Based on Siloxanes (Silicone)

3.0% by weight hexamethyldisiloxanes (proprietory name SILMOGEN CARRIER from DOW Corning) and 1% by weight ethanol were dissolved in separate mixtures by stirring in pure D₂O, in pure H₂O or in a mixture of D₂O and H₂O (1:1). These solutions were then immediately mixed in the ratio 1:2 by weight (silicone solution: carbopol gel) with vigorous stirring with the gel (carbopol gel) produced in Example 1 until optically transparent gels (silicone gels) (D₂O silicone gel, H₂O silicone gel, D₂O/H₂O silicone gel) resulted. The gels were stored at room temperature until used further, for at least 24 hours.

### Example 11

### Production of Hydrogel Based on Alginates

2.0% by weight sodium alginate (Na alginate) (manufacturer: Röhm GmbH Darmstadt, Germany) and 0.1% by weight sorbic acid were dispersed in separate mixtures by stirring in pure D₂O, in pure H₂O or in a mixture of D₂O and H₂O, and then titrated to a pH of 7.0 by pipeting 10 M NaOH solution. The resulting yellowish brown transparent gels (alginate gels) (D₂O alginate gel, H₂O alginate gel, D₂O/H₂O alginate gel) were stored at room temperature until used further, for at least 24 hours.

### Example 12

### Production of Hydrogel Based on PVA

20% by weight polyvinyl alcohol (PVA C-25, Shin-Etsu Chemical Co., Japan) was dissolved in separate mixtures by stirring in pure D₂O, in pure H₂O or in a (1:1) mixture of D₂O and H₂O. The solutions were then subjected to 5 freeze-thaw cycles. The result was gels (PVA gels) (D₂O PVA gel, H₂O PVA gel, D₂O/H₂O PVA gel) with rubbery properties which were cut into slices 2 mm thick. The gels were stored at room temperature until used further, for at least 24 hours.

### Example 13

### Production of Hydrogel Films and Sheets Based on Agarose

3.0% by weight agarose, mixed with 0.1% by weight sorbic acid, was dissolved in separate mixtures in pure D₂O, in pure H₂O or in a (1:1) mixture of D₂O and H₂O, and the solutions were then heated to 90° C. The D₂O from Sigma-Aldrich (Munich, Germany) used had an isotopic purity of 98.5%. The hot solutions were poured into suitable Petri dishes to a level of 1.0-1.5 mm and cooled. The gels obtained in this way (agarose gels) (D₂O agarose gels, H₂O agarose gels, D₂O/H₂O agarose gels) were stored under sterile conditions at 4° C.

### Example 14

### Production of Hydrogel Films or Sheets Based on Acrylamide

Acrylamide gels (5% acrylamide) were produced, with in separate mixtures pure D₂O, pure H₂O or a (1:1) mixture of D₂O and H₂O being degassed before addition of the acrylamide (which contained 2.4% bisacrylamide) and being heated to 40° C. After addition of acrylamide and bisacrylamide, the solutions were mixed (vortex mixer, 1 min at 200 rpm) and then the catalysts tetramethylethylenediamine (TEMES; 1.0%) and ammonium persulfate (AP; 0.1%) were added, followed by mixing for 10 sec. The gels were then poured into Petri dishes (height of the gel 1.0-1.5 mm) and stored at 40° C. for hours. The gels (D₂O acrylamide gel, H₂O acrylamide gel, D₂O/H₂O acrylamide gel) were then washed, using the analogous water mixture as for hydration of the gel (pure D₂O, pure H₂O or a mixture of D₂O and H₂O) for the washing. The gels were stored at room temperature until used further, for at least 24 hours.

### Example 15

### Production of a D₂O-Containing Cream

D₂O was slowly added to 50 grams of Asche® base cream (manufacturer: Asche Chiesi GmbH, Hamburg, Germany) at 40° C. while stirring continuously until the content of D₂O in the homogeneous mixture reached 45% by weight (based on the initial weight of the cream). The cream was then cooled to room temperature and stored sealed air-tight.

### Example 16

### Efficacy of D₂O Hydrogel for the Therapy of Acne Vulgaris in a Clinical Trial, Comparison with H₂O Placebo

32 healthy volunteers 15-24 years of age (16 female, 16 male) were selected for this study. All the selected test subjects had a history of acne vulgaris in the dorsal region. All the test subjects showed moderately severe acne vulgaris in the dorsal region, both with inflammatory (papules and papulopustules) and with non-inflammatory (comedones) regions. None of the test subjects used antibacterial therapeutics or immunomodulating therapeutics for one week before or during the test, which was limited to 40 days.

All the test subjects were provided with 2 gels: these were the gels based on acrylic acid produced as in Example 9, the test gel having been produced with D₂O and the placebo gel with H₂O. To avoid mixups, the placebo gel had been marked with a food dye. The test subjects were instructed to apply the gels, or have them applied, thinly on the back around a previously designated site (water-resistant marking on the skin) in a radius of at least 10 cm in each case, morning and evening, the test gel around a mark on the left side of the back and the placebo gel around a mark on the right side of the back. These marked sites (in each case one on the left and one on the right half of the back) were selected at the start of the clinical trial because of their particularly high density of papules and papulopustules. The test subjects were assessed by a dermatologist every 5 days. This entailed determination of the number of papules and papulopustules, and the number of comedones on a square area 10×10 cm in size around the center of the sites with a water-resistant marking on the left and right half of the back. This determination took place by placing an appropriately sized Plexiglas frame always on the same site (non-washable marking on the skin coinciding with marking on the Plexiglas) for each test subject and each assessment. Objects lying at the edge of the frame were also counted if at least 50% of their area was within the evaluation region.

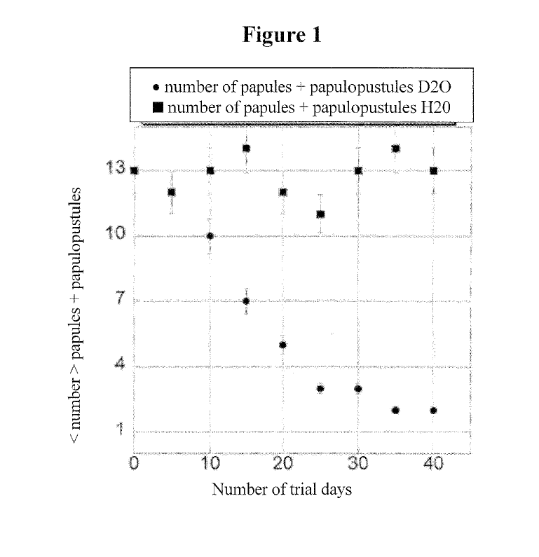

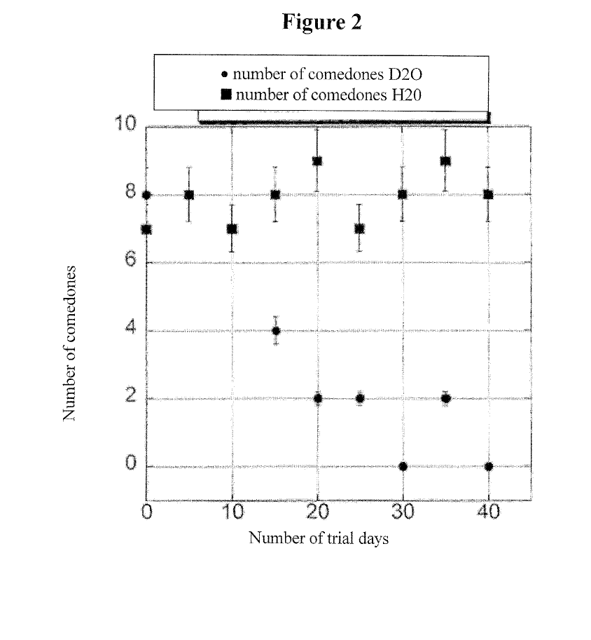

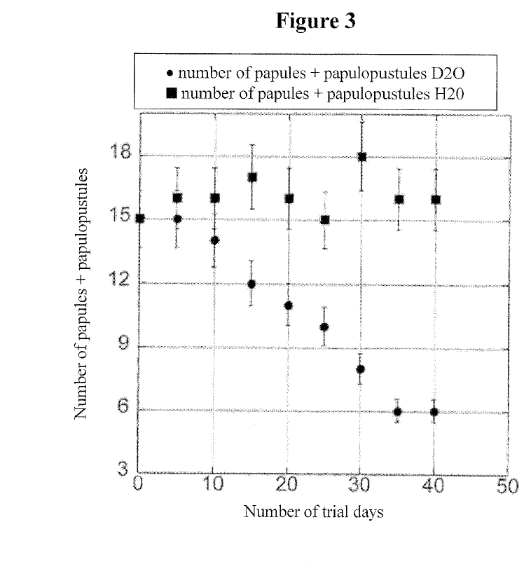

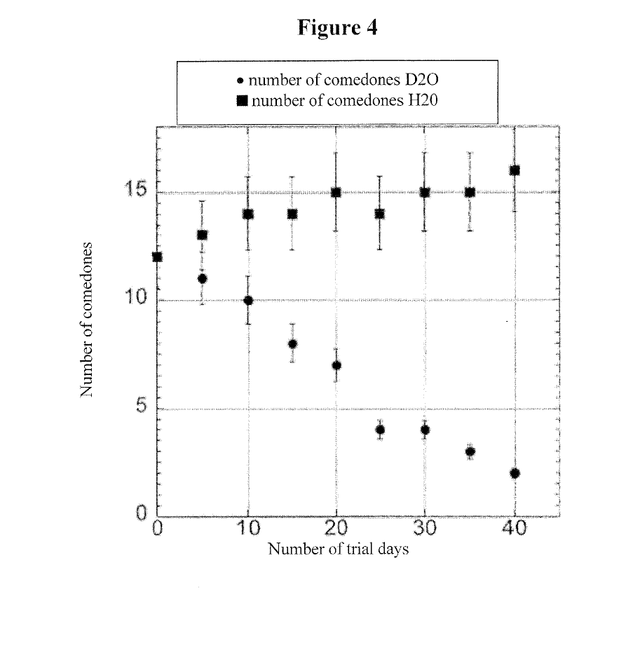

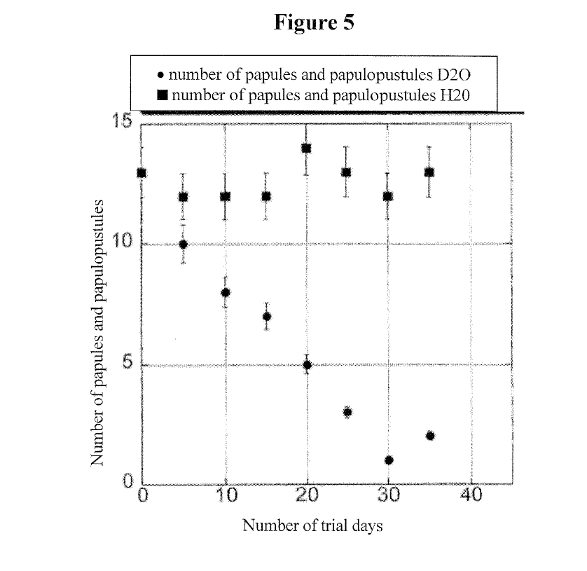

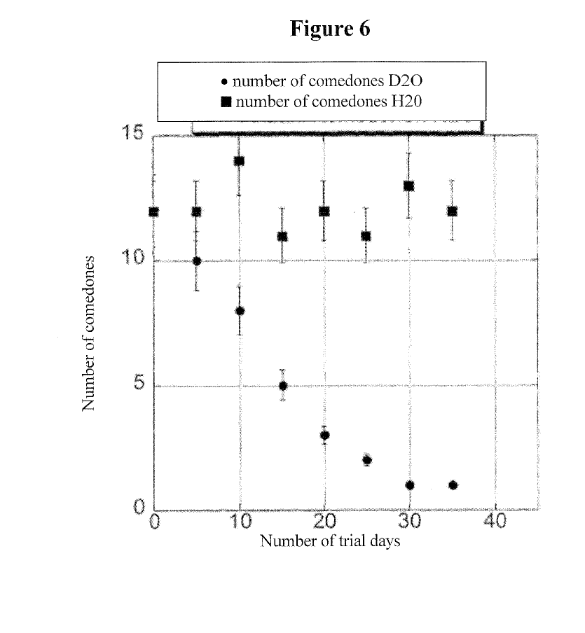

The results are shown graphically in FIG. 1 for papules and papulopustules and in FIG. 2 for comedones.

It is possible in summary to conclude from the results that D₂O gel has a clear and persistent effect in suppressing inflammatory and non-inflammatory regions of the acne vulgaris by comparison with placebo (H₂O). Whereas the difference in effect is only small in the first 10 days after the start of therapy, it becomes more distinct after the 15^{th} day of therapy and is very distinct after the 20 day of therapy. It emerges in particular that inflammatory regions in the final stage (burst follicle) are influenced only slightly, whereas the formation of new papules and papulopustules and of new comedones is significantly reduced.

### Example 17

### Efficacy of D₂O Cream for the Therapy of Acne Vulgaris in a Clinical Trial, Comparison with H₂O Placebo

14 healthy volunteers 16-24 years of age (6 female, 8 male) were selected for this study. All the selected test subjects had a history of acne vulgaris in the dorsal region. All the test subjects showed moderately severe acne vulgaris in the dorsal region, both with inflammatory (papules and papulopustules) and with non-inflammatory (comedones) regions. None of the test subjects used antibacterial therapeutics or immunomodulating therapeutics for one week before or during the test, which was limited to 40 days.

All the test subjects were provided with 2 creams: this was the cream prepared as in Example 15 based on an Asche base cream, the test cream having been produced with D₂O and the placebo cream with H₂O. To avoid mixups, the placebo cream had been marked with a food dye. The test subjects were instructed to apply the cream, or have it applied, thinly on the back around a previously designated site (water-resistant marking on the skin) in a radius of at least 10 cm in each case, morning and evening, the test cream around a mark on the left side of the back and the placebo cream around a mark on the right side of the back. These marked sites (in each case one on the left and one on the right half of the back) were selected at the start of the clinical trial because of their particularly high density of papules and papulopustules. The test subjects were assessed by a dermatologist every 5 days. This entailed determination of the number of papules and papulopustules, and the number of comedones on a square area 10×10 cm in size around the center of the sites with a water-resistant marking on the left and right half of the back. This determination took place by placing an appropriately sized Plexiglas frame always on the same site (non-washable marking on the skin coinciding with marking on the Plexiglas) for each test subject and each assessment. Objects lying at the edge of the frame were also counted if at least 50% of their area was within the evaluation region.

The results are shown graphically in FIG. 3 for papules and papulopustules and in FIG. 4 for comedones.

### Example 18

### Efficacy of D₂O Hydrogel Films for the Therapy of Acne Vulgaris in a Clinical Trial, Comparison with H₂O Placebo

10 healthy volunteers 15-24 years of age (6 female, 4 male) were selected for this study. All the selected test subjects had a history of acne vulgaris in the dorsal region. All the test subjects showed moderately severe acne vulgaris in the dorsal region, both with inflammatory (papules and papulopustules) and with non-inflammatory (comedones) regions. None of the test subjects used antibacterial therapeutics or immunomodulating therapeutics for one week before or during the test, which was limited to 40 days.

Agarose hydrogel films produced as in Example 13 with a thickness of 2 mm, either from D₂O (test gel) or H₂O (placebo gel) base, were cut into pieces 10×10 cm in size and placed on previously marked sites (water-resistant marking) on the backs, centered on the marked site, of the test subjects, and fixed with Tegaderm plaster. These marked sites (in each case one on the left and one on the right half of the back) were selected at the start of the clinical trial because of their particularly high density of papules and papulopustules. The test gel was applied to the left half of the back and the placebo gel to the right half of the back. The test subjects were assessed by a dermatologist every 5 days and then the agarose gel was replaced by a new gel of the same size in each case. This entailed determination of the number of papules and papulopustules, and the number of comedones on a square area 10×10 cm in size around the center of the sites with a water-resistant marking on the left and right half of the back. This determination took place by placing an appropriately sized Plexiglas frame (after removal of the agarose gel) always on the same site (non-washable marking on the skin coinciding with marking on the Plexiglas) for each test subject and each assessment. Objects lying at the edge of the frame were also counted if at least 50% of their area was within the evaluation region.

The results are shown graphically in FIG. 5 for papules and papulopustules and in FIG. 6 for comedones.

### Example 19

### Efficacy of D₂O Cream for Therapy of Diabetic Foot (Neuoropathically Infected Foot) in a Clinical Trial, Comparison with H₂O Placebo

12 volunteers 25-62 years of age (6 female, 6 male) were selected for this study. All the selected test subjects had a history of diabetes mellitus (type 1 or 2) and had pronounced symptoms of the neuropathically infected diabetic foot. All the test subjects were characterized by an extensive callosity on the soles of the feet.

All the test subjects were provided with 2 creams: this was the cream produced as in Example 15 based on an Asche base cream, the test cream having been produced with D₂O and the placebo cream with H₂O. To avoid mixups, the placebo cream had been marked with a food dye. The test subjects were instructed to apply the cream, or have it applied, thinly to the most affected foot around two sites previously identified in each case (water-resistant marking on the callosity) in a radius of at least 2 cm morning and evening. The markings were different in color, and the test subjects were instructed to apply the test cream around the red marking and the placebo cream around the black marking. The sites with water-resistant markings were selected before the start of the trial because of their particularly thick callosity. An assessment by a dermatologist took place every days during the clinical trial which lasted a total of 40 days.

The results can be summarized as follows. After 10 days from the start of the trial (second dermatological assessment) it was not possible to observe any significant differences in the callosity around the two markings. After 20 days from the start of the trial there were visible signs of the callosity growing again more slowly for the test cream than for the placebo cream, and this was clearly visible at the fourth dermatological assessment on day 30 after the start of the trial, both on the basis of the elasticity of the skin and of its color. On day 40 after the start of the trial there were significant differences visible between the two treated areas which can be explained by a reduced thickness of the callous layer of the regions treated with the test cream compared with the placebo cream.

1-39. (canceled)

40. A method for the prophylaxis and/or therapy of a hyperproliferative skin disease wherein the method comprises administering to a subject in need of such treatment D₂O, and wherein the hyperproliferative skin disease is a non-malignant disease of the skin.

41-43. (canceled)

44. The method, according to claim 40, wherein the non-malignant skin disease is selected from psoriasis, keratoses and skin scars.

45. The method, according to claim 44, wherein the psoriasis is selected from psoriasis vulgaris, psoriasis guttata, psoriasis inversa, psoriasis capitis, psoriasis pustulosa and psoriatic arthritis.

46. The method, according to claim 44, wherein the keratosis is selected from benign lichenoid keratosis, palmoplantar keratosis, follicular keratosis, verruca seborrhoica and lichen-planus-like keratosis, porokeratosis, actinic keratosis, epidermolytic hyperkeratosis, hyperkeratosis lenticularis perstans, keratosis pilaris, ichthyosis, acne and hyperkeratosis in connection with diabetes mellitus.

47. The method, according to claim 46, wherein the porokeratosis is selected from porokeratosis disseminata, porokeratosis mibelli, porokeratosis naeviformis, porokeratosis striata, and porokeratosis disseminata.

48. The method, according to claim 46, wherein the acne is selected from acne vulgaris, acne inversa, acne comedonica, acne papula-pustulosa, acne conglobata, hidradentis suppurativa, acne aestivalis, acne cosmetica, acne medicamentosa, acne venenata and acne tarda.

49. The method, according to claim 44, wherein the skin scars are selected from hypertrophic scars and keloids.

50. The method, according to claim 40, wherein the D₂O, is applied topically to skin.

51. The method according to claim 40, wherein the D₂O suppresses and/or inhibits proliferation of skin cells.

52. The method, according to claim 51, wherein the skin cells are selected from the group consisting of keratinocytes, epidermal cells, dermal cells, fibroblasts, collagen cells, connective tissue cells and melanocytes.

53. The method, according to claim 40, wherein the D₂O is used in combination with at least one additional pharmaceutical ingredient and/or at least one additional non-pharmaceutical ingredient.

54. The method, according to claim 53, wherein the at least one additional pharmaceutical ingredient is selected from the group consisting of cytostatics, proteins, peptides, nucleic acids, immunosuppressive agents, and growth factors.

55. The method, according to claim 53, wherein the at least one additional non-pharmaceutical ingredient is selected from the group consisting of pharmaceutically tolerable inorganic or organic acids or bases, polymers, copolymers, block copolymers, monosaccharides, polysaccharides, ionic and non-ionic tensides or lipids, as well as mixtures thereof; albumin, transferrin and DNA repair proteins.

56. The method, according to claim 40, wherein the D₂O is applied topically with a plaster or a bandage.

57. The method, according to claim 56, wherein the plaster or the bandage is used in combination with at least one membrane or at least one film.

58. The method, according to claim 57, wherein the membrane is a microporous or nanoporous membrane.

59. The method, according to claim 56, wherein the film is a microporous or nanoporous film.

60. A plaster or bandage, intended for topical application, and which contains D₂O.

61. The plaster or bandage, according to claim 60, wherein, the plaster or the bandage contains at least one additional pharmaceutical ingredient and/or at least one additional non-pharmaceutical ingredient.

62. The plaster or bandage, according to claim 61, wherein the at least one additional pharmaceutical ingredient is selected from the group consisting of cytostatics, proteins, peptides, nucleic acids, immunosuppressive agents, and growth factors.

63. The plaster or bandage, according to claim 60, wherein the at least one additional non-pharmaceutical ingredient is selected from the group consisting of pharmaceutically tolerable inorganic or organic acids or bases, polymers, copolymers, block copolymers, monosaccharides, polysaccharides, ionic and non-ionic tensides or lipids, as well as mixtures thereof; albumin, transferrin and DNA repair proteins.

64. The method, according to claim 40, wherein the D₂O is applied as an aerosol.

65. An aerosol, which comprises a mixture of D₂O and H₂O for topical application on the skin.

66. The aerosol, according to claim 65, wherein the aerosol further comprises at least one additional pharmaceutical ingredient and/or at least one additional non-pharmaceutical ingredient.

67. The aerosol, according to claim 66, wherein the at least one additional pharmaceutical ingredient is selected from the group consisting of cytostatics, proteins, peptides, nucleic acids, immunosuppressive agents, and growth factors.

68. The aerosol, according to claim 65, wherein the at least one additional non-pharmaceutical ingredient is selected from the group consisting of pharmaceutically tolerable inorganic or organic acids or bases, polymers, copolymers, block copolymers, monosaccharides, polysaccharides, ionic and non-ionic tensides or lipids, as well as mixtures thereof; albumin, transferrin and DNA repair proteins.

69. The aerosol, according to claim 65, which further comprises an inorganic or organic solvent.

70. The aerosol, according to claim 69, wherein the solvent is selected from the group consisting of ethanol, water, glycerol and mixtures thereof.

71. The method, according to claim 40, wherein D₂O is applied topically as a formulation.

72. A formulation, which is intended for topical application on the skin and contains D₂O.

73. The formulation, according to claim 72, wherein the formulation is an ointment, cream or gel.

74. The formulation, according to claim 72, wherein the formulation contains at least one additional pharmaceutical ingredient and/or at least one additional non-pharmaceutical ingredient.

75. The formulation, according to claim 74, wherein the at least one additional pharmaceutical ingredient is selected from the group consisting of cytostatics, proteins, peptides, nucleic acids, immunosuppressive agents, and growth factors.

76. The formulation, according to claim 74, wherein the at least one additional non-pharmaceutical ingredient is selected from the group consisting of pharmaceutically tolerable inorganic or organic acids or bases, polymers, copolymers, block copolymers, monosaccharides, polysaccharides, ionic and non-ionic tensides or lipids, as well as mixtures thereof; albumin, transferrin and DNA repair proteins.

77. The formulation, according to claim 62, which further comprises at least one inorganic or organic solvent.

78. The formulation, according to claim 77, wherein the solvent is selected from the group, consisting of ethanol, water, glycerol and mixtures thereof.
